(19) United States
(12) Patent Application Publication
(10) Pub. No.: US 2009/0253138 A1
(43) Pub. Date: Oct. 8, 2009

(54) KIF20A DIRECTED DIAGNOSTICS FOR NEOPLASTIC DISEASE

(76) Inventors: Elias GEORGES, Laval (CA); Anne-Marie BONNEAU, Laval (CA)

Correspondence Address: WILMERHALE/BOSTON, 60 STATE STREET, BOSTON, MA, 02109, US

(21) Appl. No.: 12/361,787

(22) Filed: Jan. 29, 2009

Related U.S. Application Data
(60) Provisional application No. 61/024,430, filed on Jan. 29, 2008.

(51) Int. Cl. C12Q 1/68; G01N 33/53

(52) U.S. Cl. 435/6; 435/7.1

(57) ABSTRACT

Disclosed are methods for diagnosing cancer in a test cell sample or fluid sample by detecting an increase in the level of expression of KIF20A in the test cell sample or fluid sample as compared to the level of expression of KIF20A in a control cell sample or fluid sample isolated from a normal subject.

### CROSS REFERENCE TO RELATED APPLICATIONS

This application claims the benefit of U.S. Provisional Patent Application Ser. No. 61/024,430 of Elias Georges, et al. entitled “KIF20A Directed Diagnostics and Neoplastic Disease,” filed Jan. 29, 2008. The entirety of the provisional patent application is incorporated herein by reference.

### FIELD OF THE INVENTION

The present invention relates generally to the field of medicine. More specifically, the invention pertains to methods and kits for detecting the development of cancer in a subject.

### BACKGROUND OF THE INVENTION

Cancer is one of the most deadly illnesses in the world. It accounts for nearly 600,000 deaths annually in the United States, and costs billions of dollars for those who suffer from the disease. This disease is in fact a diverse group of disorders, which can originate in almost any tissue of the body. In addition, cancers may be generated by multiple mechanisms including pathogenic infections, mutations, and environmental insults. The variety of cancer types and mechanisms of tumorigenesis add to the difficulty associated with treating a tumor, increasing the risk posed by the cancer to the patient's life and well-being.

Cancers manifest abnormal growth and the ability to move from an original site of growth to other tissues in the body (“metastasis”), unlike most non-cancerous cells. These clinical manifestations are therefore used to diagnose cancer because they are applicable to all cancers. Additionally, a cancer diagnosis is made based on identifying cancer cells by their gross pathology through histological and microscopic inspection of the cells. Although the gross pathology of the cells can provide accurate diagnoses of the cells, the techniques used for such analysis are hampered by the time necessary to process the tissues and the skill of the technician analyzing the samples. These methodologies can lead to unnecessary delay in treating a growing tumor, thereby increasing the likelihood that a benign tumor will acquire metastatic characteristics. It is thus necessary to accurately diagnose potentially cancerous growths in early stages to avoid the development of a potentially life threatening illness.

One potential method of increasing the speed and accuracy of cancer diagnoses is the examination of genes as markers for neoplastic potential. Recent advances in molecular biology have identified genes involved in cell cycle control, apoptosis, and metabolic regulation (see, e.g., Isoldi et al. (2005) Mini Rev. Med. Chem. 5(7): 685-95). Mutations in many of these genes have also been shown to increase the likelihood that a normal cell will progress to a malignant state (see, e.g., Soejima et al. (2005) Biochem. Cell Biol. 83(4): 429-37). Many mutations can affect the levels of expression of certain genes in the neoplastic cells as compared to normal cells.

There remains a need to identify an accurate and rapid means for diagnosing cancer in patients. Treatment efficacy would be improved by more efficient diagnoses in fluid (e.g., blood) or tissue samples. Furthermore, rapid diagnoses of cancerous tissues or blood samples from patients may allow clinicians to treat potential tumors prior to the metastasis of the cancer to other tissues of the body. Finally, a test that does not rely upon a particular technician's skill at identifying abnormal histological characteristics would improve the reliability of cancer diagnoses. There is, therefore, a need for new methods of diagnoses for cancer that are accurate, fast, and relatively easy to interpret. In addition, such tests are useful to follow the response of patients to cancer treatment.

### SUMMARY OF THE INVENTION

The present invention is based in part upon the discovery that differential expression of KIF20A (Kinesin Family Member 20A; or “KIF20A”) at the protein and RNA levels occurs when a cell progresses to a neoplastic state. These expression patterns are therefore diagnostic for the presence of cancer in a cell sample. This discovery has been exploited to provide an invention that uses such patterns of expression to diagnose the presence of neoplastic cells in the test sample (cell sample or fluid sample, where the protein is secreted or released in circulation). The KIF20A may be found as full length protein and/or peptides or fragments of KIF20A. Similarly, a test sample may contain the KIF20A RNA or modified nucleotide fragments of this gene.

Accordingly, in one aspect, the invention provides a method of detecting a neoplasm comprising: a) obtaining a potentially neoplastic test sample and a corresponding non-neoplastic control sample; b) detecting a level of KIF20A expression in the test sample and in the control sample; and c) comparing the level of KIF20A expression in the test sample to the level of KIF20A expression in the control sample. The test sample is neoplastic if the level of KIF20A expression in the test sample is detectably greater than the level of KIF20A expression in the control sample.

In some embodiments, the level of expression of KIF20A protein is detected by contacting the test sample and the control sample with a KIF20A-specific protein binding agent selected from the group consisting of an anti-KIF20A antibody, KIF20A-binding portions of an antibody, KIF20A-specific ligands, KIF20A-specific aptamers, and KIF20A inhibitors. In certain embodiments, KIF20A-specific binding agent bound to KIF20A protein further comprises a detectable label. In particular embodiments, the detectable label is selected from the group consisting of an immunofluorescent label, a radiolabel, and a chemiluminescent label.

In some embodiments, the KIF20A-specific protein binding agent is immobilized on a solid support.

In other embodiments, KIF20A expression is detected by detecting the level of expression of KIF20A RNA by contacting the test sample and the control sample with a KIF20A RNA-specific nucleic acid binding agent and determining how much of the nucleic acid binding agent is hybridized to KIF20A RNA in the test sample and in the control sample. In some embodiments, the level of nucleic acid binding agent hybridized to KIF20A RNA is detected using a detectable label operably linked to the binding agent. In particular embodiments, the label is selected from the group consisting of an immunofluorescent label, a radiolabel, and a chemiluminescent label. In certain embodiments, the nucleic acid binding agent is immobilized on a solid support.

In some embodiments, the level of expression of KIF20A in the test sample is at least 1.5 times greater, at least 2 times greater, at least 4 times greater, at least 6 times greater, at least 8 times greater, at least 10 times greater, or at least 20 times greater than the level of expression of KIF20A in the control sample. In certain embodiments, the test sample is isolated from a patient suffering from ovarian cancer, breast cancer, colon cancer, lung cancer, melanoma, sarcoma, or leukemia, and in some embodiments, the cancer is a metastacized cancer.

In particular embodiments, neoplastic test sample and the control samples are cell samples of the same lineage. In certain embodiments, a cytoplasmic fraction is isolated from the test cell sample and from the control cell sample, and then the level of expression of KIF20A in each of these cytoplasmic fractions is detected separately.

In other embodiments, the test sample and the control samples are fluid samples. In certain embodiments, the fluid samples are blood, serum, urine, seminal fluid, lacrimal secretions, sebaceous gland secretions, tears, or vaginal secretions. In a particular embodiment, the fluid sample is a serum sample. In some embodiments, the level of KIF20A protein expression is determined by measuring the level of anti-KIF20A antibody in the test fluid sample and in the control fluid sample. In certain embodiments, the level of expression of anti-KIF20A antibody is detected with an anti-KIF20A antibody-specific antibody, or anti-KIF20A antibody-specific antibody fragment thereof. In some embodiments, the anti-KIF20A antibody-specific antibody, or anti-KIF20A antibody-specific binding fragments thereof, are operably linked to a detectable label.

In another aspect, the invention provides a method for detecting a neoplasm comprising: a) obtaining a potentially neoplastic test sample and a non-neoplastic control sample; b) detecting a level of KIF20A expression in the test sample and in the control sample; c) detecting a level of expression of at least one of TRIM59, TTK, SLC7A5, and/or UHRF1; and d) comparing the level of KIF20A expression and the level of expression of at least one of TTK, SLC7A5, TRIM59 and/or UHRF1 in the test sample to the level of KIF20A expression and the level of expression of the at least one of TTK, SLC7A5, TRIM59 and/or UHRF1 in the control sample. The test sample is neoplastic if the levels of expression of KIF20A and the at least one of TTK, SLC7A5, TRIM59 and/or UHRF1 in the test sample are detectably greater than the levels of expression of UHRF1 and the at least one of TTK, SLC7A5, TRIM59 and/or KIF20A in the control sample. In some embodiments, detecting step (c) comprises detecting the level of at least UHRF1 and/or TTK expression, and comparing step (d) comprises comparing the levels of expression of KIF20A and at least UHRF1 and/or TTK expression in the test and control samples.

In some embodiments, the level of KIF20A expression is detected by contacting the test sample and the control sample with a KIF20A-specific protein binding agent selected from the group consisting of an KIF20A-specific antibody, KIF20A-specific binding portions of an antibody, a KIF20A-specific ligand, a KIF20A-specific aptamer, and an KIF20A inhibitor. In certain embodiments, the KIF20A-specific protein binding agent is immobilized on a solid support.

In other embodiments, the level of expression of KIF20A in the test and control samples is measured by measuring the level of KIF20A RNA and the level of at least one of TTK RNA, SLC7A5 RNA, TRIM59 RNA, and/or UHRF1 RNA in the test and control samples. In some embodiments, the level of expression of KIF20A RNA and the level of expression of at least one of TTK RNA, SLC7A5 RNA, TRIM59 RNA, and/or UHRF1 RNA are detected by contacting the test sample and the control sample with an KIF20A-specific nucleic acid binding agent and with at least one of a TTK-specific nucleic acid binding agent, a SLC7A5-specific nucleic binding agent, a TRIM59-specific nucleic acid binding agent, and a UHRF1-specific nucleic acid binding agent.

In some embodiments, the levels of expression of UHRF1, TTK, SLC7A5, TRIM59 and/or KIF20 in the test sample are at least about 1.5, 2, 5, 10, or 20 times greater than the level of expression of UHRF1, TTK, SLC7A5, TRIM59, and/or KIF20 in the control sample.

In particular embodiments, detecting the level of expression of KIF20A and the level of expression of at least one of TTK, SLC7A5, TRIM59 and/or UHRF1 comprises isolating a cytoplasmic fraction from the test cell sample and from the control cell sample, and then detecting the levels of expression of KIF20A and at least one of TTK, SLC7A5, TRIM59 and/or UHRF1 in each of these cytoplasmic fractions.

In some embodiments, the test and control samples are fluid samples, and in certain embodiments, the level of expression of KIF20A is measured by detecting a level of anti-KIF20A antibody in a test fluid sample and in a control fluid sample.

In certain embodiments, the test sample is isolated from a tissue of a patient suffering from ovarian cancer, breast cancer, lung cancer, sarcoma, melanoma, or leukemia. In particular embodiments, the cancer has metasticized.

In yet another aspect, the invention provides a kit for diagnosing or detecting neoplasia. The kit comprises: a) a first probe specific for the detection of KIF20A; and b) a second probe specific for the detection of a neoplasia marker selected from the group consisting of TTK, SLC7A5, TRIM59, UHRF1, and combinations thereof.

In some embodiments, the probe for detecting KIF20A is an anti-KIF20A-specific antibody or an KIF20A-specific binding fragment thereof, a KIF20A-specific aptamer, or KIF20A-specific ligand.

In some embodiments, the second probe is selected from the group consisting of a TTK-specific antibody, a TTK-specific binding portion of TTK antibody, a TTK-specific ligand, a TTK-specific aptamer, a SLC7A5-specific antibody, a SLC7A5-specific binding portion of a SLC7A5-specific antibody, a SLC7A5-specific ligand, a SLC7A5-specific aptamer, a TRIM59-specific antibody, a TRIM59-specific binding portion of a TRIM59-specific antibody, a TRIM59-specific ligand, a TRIM59-specific aptamer, a UHRF1-specific binding portion of a UHRF1-specific antibody, a UHRF1-specific ligand, a UHRF1-specific aptamer, and combinations thereof.

In other embodiments, the first probe for detecting KIF20A is a KIF20A RNA-specific nucleic acid binding agent. In certain embodiments, the second probe is selected from the group consisting of an SLC75A-specific nucleic acid RNA-binding agent, a TTK RNA-specific nucleic acid binding agent, a TRIM59 RNA-specific nucleic acid binding agent, a UHRF1 RNA-specific nucleic acid binding agent, and combinations thereof. In some embodiments, the kit further comprising a solid support to which the first probe and/or the second probe(s) is/are immobilized or can be immobilized. In certain embodiments, the first probe and/or the second probe is selected from the group consisting of RNA, cDNA, cRNA, and RNA-DNA hybrids. In particular embodiments the KIF20A probe is complementary to at least a 20 nucleotides of a nucleic acid sequence consisting of SEQ ID NO: 6. In some embodiments, the second probe is a nucleic acid probe complementary to at least a 20 nucleotide sequence of a nucleic acid sequence selected from the group consisting of SEQ ID NOS: 7, 8, 9, and 10. the first probe and/or the second probe further comprises a detectable label in some embodiments.

### BRIEF DESCRIPTION OF THE FIGURES

The foregoing and other objects of the present invention, the various features thereof, as well as the invention itself may be more fully understood from the following description, when read together with the following accompanying drawings:

FIG. 1 is a graphic representation of the differential expression of KIF20A RNA in NSCLC tumors relative to normal lung samples from patients as measured by qRT-PCR where results are expressed as normalized ratio of KIF20A between patients' samples and H23 tumor lung cell line calibrator. The results shown in this figure are based on sample size of NSCLC patients n=11 and Normal patient n=15. Unpaired Student's t test was done and equal to p=0.0088.

FIG. 2 is a graphic representation of the differential expression of KIF20A in breast, ovarian, colorectal and prostate cancers, relative to normal samples in these tissues as measured by qRT-PCR, where results are expressed as normalized ratio of KIF20A between patients' samples from breast, ovarian, colorectal and prostate samples and H23 tumor lung cell line calibrator. The results shown in this figure are based on sample size of NSCLC (n=15N+11T); Breast (N=10N+17T); Ovarian (n=10N+17T); Colorectal (n=10N+10T matched); Prostate (n=10N+10T matched).

FIG. 3 is a graphic representation of a ROC curve for KIF20A in lung cancer, where the large dashed lines represent 95% confidence limits and are based on a group of normal and NSCLC samples (n=15N+11T).

FIG. 4 is a graphic representation of the differential expression of KIF20A RNA in breast cancer, where results are expressed as normalized ratio of KIF20A between patients' samples and H23 tumor cell line calibrator. A 13.1 fold increase was obtained between the tumor and the normal patients. Breast cancer patients n=17; Normal patient n=10. Unpaired Student's t test was done and p<0.0001.

FIG. 5 is a graphic representation of the differential expression of KIF20A RNA in different stage breast cancer tumors, where results are expressed as normalized ratio of KIF20A RNA expression between patient samples and H23 tumor cell line calibrator. Breast cancer patients at stage 1 (n=7) and stage 2 (n=10) were compared to normal breast samples (n=10). Non-parametric Kiruskal-Wallis test (p=0.0001) with Dunn's multiple comparison test was run to assess the significance of KIF20A expression between normal and stage I breast cancer patients (p<0.01); normal and stage II breast cancer patients (p<0.001) and between stage I and stage 11 breast cancer patients.

FIG. 6 is a graphic representation of ROC curves for KIF20A in breast cancer, where the large dashed lines represent 95% confidence limits and are based on a group of normal and breast cancer samples (N=10N+17T).

FIG. 7 is a graphic representation of the differential expression of KIF20A RNA in ovarian cancer, where results are expressed as normalized ratio of KIF20A between patients' samples and H23 tumor cell line calibrator. A 2.9 fold increase in the expression of KIF20A RNA was observed in ovarian cancer samples relative to normal samples. Ovarian cancer patients n=17 (n=8 stage I/II; n=9 stage III); Normal patient n=10. Unpaired Student's t test was done and p=0.0193.

FIG. 8 is a graphic representation of ROC curves for KIF20A in ovarian cancer, where the large dashed lines represent 95% confidence limits and are based on a group of normal and ovarian cancer samples (N=10N+17T).

FIG. 9 is a graphic representation of the differential expression of KIF20A RNA in colorectal cancer, where results are expressed as normalized ratio of KIF20A between patients' samples and H23 tumor cell line calibrator. A 7.1 fold increase in the expression of KIF20A RNA was observed in colorectal cancer samples relative to normal samples. Colorectal cancer patients n=10; normal matched samples n=10. Unpaired Student's t test was done and p=0.0016.

FIG. 10 is a graphic representation of ROC curves for KIF20A in colorectal cancer, where the large dashed lines represent 95% confidence limits and are based on a group of normal and colorectal cancer samples (n=10N+10T matched).

FIG. 11 is a representation of representative nucleotide sequences for KIF20A, UHRF1, TTK, TRIM59, and SLC7A5.

FIG. 12 is a representation of representative amino acid sequences for KIF20A, UHRF1, TTK, TRIM59, and SLC7A5.

### DETAILED DESCRIPTION OF THE INVENTION

Patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.

### 1.1 General

The present invention provides, in part, methods and kits for diagnosing, detecting, or screening a test sample, such as a fluid or cell sample, for tumorigenic potential and neoplastic characteristics such as aberrant growth. The invention also allows for the improved clinical treatment and management of tumors by providing a method that detects the expression level of a gene or genes identified as markers for cancer. One such gene expresses the biomarker KIF20A.

Typically, a gene will affect the phenotype of the cell through its expression at the protein level. Mutations in the coding sequence of the gene can alter its protein product in such a way that the protein does not perform its intended function appropriately. Some mutations, however, affect the levels of protein expressed in the cell without altering the functionality of the protein, itself. Such mutations directly affect the phenotype of a cell by changing the delicate balance of protein expression in a cell. Therefore, an alteration in a gene's overall activity can be measured by determining the level of expression of the protein product of the gene in a cell.

Accordingly, one aspect of the invention provides a method for diagnosing cancer in a cell. The method utilizes protein-targeting agents to identify protein markers, such as KIF20A, in a potentially cancerous cell sample or potentially cancerous fluid sample. Increased levels of expression of particular protein markers in a cell or fluid sample and a decreased expression level of other protein markers in a cell or fluid sample indicate the presence of a neoplasm.

KIF20A belongs to the family of kinesin proteins, which are characterized by a conserved motor domain that binds to microtubules and couples ATP hydrolysis to generate mechanical force (Hirokawa et al., 1998; Echard et al., 1998). KIF20A play a role in the dynamics of the Golgi apparatus through direct interaction with Rab6 small GTPase (Echard et al., 1998). It also acts as a motor required for the retrograde rab6 regulated transport of golgi membranes and associated vesicles along microtubules. Furthermore, KIF20A is implicated in protein transport, vesicle-mediated transport, microtubule-based movement, in addition to its role in cytokinesis (Hill et al., 2000; Fontijn et al., 2001). KIF20A is highly expressed in fetal tissues, adult thymus, bone marrow, and testis (Fontijn et al., 2001) and low level expression in heart, placenta, and spleen. KIF20A is expressed in brain, lung, liver, skeletal muscle, kidney, pancreas and peripheral lymphocytes.

As used herein, the term “cancer” refers to a disease condition in which a tissue or cells exhibit aberrant, uncontrolled growth and/or lack of contact inhibition. A cancer can be a single cell or a tumor composed of hyperplastic cells. In addition, cancers can be malignant and metastatic, spreading from an original tumor site to other tissues in the body. In contrast, some cancers are localized to a single tissue of the body.

As used herein, a “cancer cell” is a cell that shows aberrant cell growth, such as increased, uncontrolled cell proliferation and/or lack of contact inhibition. A cancer cell can be a hyperplastic cell, a cell from a cell line that shows a lack of contact inhibition when grown in vitro, or a cancer cell that is capable of metastasis in vivo. In addition, cancer cells include cells isolated from a tumor or tumors. As used herein, a “tumor” is a collection of cells that exhibit the characteristics of cancer cells. Non-limiting examples of cancer cells include melanoma, ovarian cancer, ovarian cancer, renal cancer, osteosarcoma, lung cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, and thymoma. Cancer cells are also located in the blood at other sites, and include, but are not limited to, lymphoma cells, melanoma cells, sarcoma cells, leukemia cells, retinoblastoma cells, hepatoma cells, renal cancer cells, osteosarcoma cells, myeloma cells, glioma cells, mesothelioma cells, and carcinoma cells.

Cancer cells may also have the ability to metastasize to other tissues in the body. “Metastasis” is the process by which a cancer cell is no longer confined to the tumor mass, and enters the blood stream, where it is transported to a second site. Upon entering the other tissue, the cancer cell gives rise to a second situs for the disease and can take on different characteristics from the original tumor. Nevertheless, the new tumor retains characteristics from the tissue from which it derives, allowing for clinical identification of the type of cancer no matter where in the body a cancer cell or group of cells metastasizes. The process of metastasis has been studied extensively and is known in the art (see, e.g., Hendrix et al. (2000) Breast Cancer Res. 2(6): 417-22). The metastasized cells may be isolated from tissues including, but not limited to, blood, bone marrow, lymph node, liver, thymus, kidney, brain, skin, gastrointestinal tract, breast, and prostate.

As used herein, the term “tumorigenic potential” means ability to give rise to either benign or malignant tumors. Tumorigenic potential may occur through genetic mechanisms such as mutation or through infection with vectors such as viruses and bacteria.

The term “protein markers” as used herein means any protein, peptide, polypeptides, group of peptides, polypeptides or proteins expressed from a gene, whether chromosomal, extrachromosomal, endogenous, or exogenous, which may produce a cancerous or non-cancerous phenotype in the cell or the organism.

Protein markers can have any structure or conformation, and can be in any location within a cell, including on the cell surface. Protein markers can also be secreted from the cell into an extracellular matrix or directly into the blood or other biological fluid. Protein markers can be a single polypeptide chain or peptide fragments of a polypeptide. Moreover, they can also be combinations of nucleic acids and polypeptides as in the case of a ribosome. Protein markers can have any secondary structure combination, any tertiary structure, and come in quaternary structures as well.

One useful protein marker used to identify a neoplastic disease is KIF20A protein. Examples of KIF20A amino acid sequences include, but are not limited to, GenBank Accession Nos. NP_{—}005724, AAH12999, EAW62158, and EAW62157. Other useful protein markers include, but are not limited to, TRIM59, TTK, SLC7A5, and UHRF1.

As used herein, “gene” means any deoxyribonucleic acid sequence capable of being translated into a protein or peptide sequence. The gene is a DNA sequence that may be transcribed into an mRNA and then translated into a peptide or protein sequence. Extrachromosomal sources of nucleic acid sequences can include double-strand DNA viral genomes, single-stranded DNA viral genomes, double-stranded RNA viral genomes, single-stranded RNA viral genomes, bacterial DNA, mitochondrial genomic DNA, cDNA or any other foreign source of nucleic acid that is capable of generating a gene product.

As used herein, the term “protein-targeting agent” or “protein binding agent” means a molecule capable of binding, interacting, or associating with a protein or a portion of a protein. Such binding or interactions can include ionic bonds, van der Waals interactions, London forces, covalent bonds, and hydrogen bonds. The target protein can be bound in a receptor-binding pocket, on its surface, or any other portion of the protein that is accessible to binding or interactions with a molecule. Protein-targeting agents include, but are not limited to, proteins, peptides, ligands, peptidomimetic compounds, inhibitors, organic molecules, aptamers, or combinations thereof.

As used herein, the term “inhibitor” means a compound that prevents a biomolecule, e.g., a protein, nucleic acid, or ribozyme, from completing or initiating a reaction. An inhibitor can inhibit a reaction by competitive, uncompetitive, or non-competitive means. Exemplary inhibitors include, but are not limited to, nucleic acids, proteins, small molecules, chemicals, peptides, peptidomimetic compounds, and analogs that mimic the binding site of an enzyme. In some embodiments, the inhibitor can be nucleic acid molecules including, but not limited to, siRNA that reduce the amount of functional protein in a cell.

As used herein, the term “greater than” means more than, such as when the level of expression for a particular marker in test sample is detectably more than the level of expression for the same marker in a control sample. In these circumstances, expression analyses are qualitatively determined. The level of expression for a marker can also be determined quantitatively in test and control samples. In quantitative studies, the level of expression for a marker in a test sample is greater than the level of expression for the same marker in a control sample when the level of expression in the test sample is quantifiably determined to be at least about 10% more than the level of expression in the control sample.

As used herein, “about” means a numeric value having a range of ±10% around the cited value. For example, a range of “about 1.5 times to about 2 times” includes the range “1.35 times to 2.2 times” as well as the range “1.65 times to 1.8 times,” and all ranges in between.

In another aspect, the invention provides methods for diagnosing cancer in a test cell sample by detecting KIF20A protein using a dipstick assay, Western blots, dot blots, and Enzyme-Linked Immunosorbent Assays (“ELISA's”).

KIF20A can also be detected with different cancer markers using a protein microarray. The methods can be practiced using a microarray composed of capture probes affixed to a derivatized solid support such as, but not limited to, glass, nylon, metal alloy, or silicon. Non-limiting examples of derivatizing substances include aldehydes, gelatin-based substrates, epoxies, poly-lysine, amines and silanes. Techniques for applying these substances to solid surfaces are well known in the art. In useful embodiments, the solid support can be comprised of nylon.

For purposes of the invention, the term “capture probe” is intended to mean any agent capable of binding a gene product in a complex cell sample or fluid sample. Capture probes can be disposed on the derivatized solid support utilizing methods practiced by those of ordinary skill in the art through a process called “printing” (see, e.g., Schena et. al., (1995) Science, 270(5235): 467-470). The term “printing”, as used herein, refers to the placement of spots onto the solid support in such close proximity as to allow a maximum number of spots to be disposed onto a solid support. The printing process can be carried out by, e.g., a robotic printer. The VersArray CHIP Writer Prosystem (BioRad Laboratories) using Stealth Micro Spotting Pins (Telechem International, Inc, Sunnyvale, Calif.) is a non-limiting example of a chip-printing device that can be used to produce a focused microarray for this aspect. The capture probes may be antibodies, fragments thereof, or any other molecules capable of binding a protein (herein termed “protein capture probes”). These probes may be attached to a solid support at predetermined positions.

### 1.2 Samples to be Tested

In the present invention, samples containing tumor cells and/or tumor cell markers are taken and screened relative to control samples. Samples can be fluid or cell samples.

As used herein, the term “fluid sample” refers to a liquid sample. Such samples can be isolated from biological fluids, e.g., urine, blood, lymph, pleural fluid, pus, marrow, cartilaginous fluid, saliva, seminal fluid, amniotic fluid, menstrual blood, lacrimal secretions, vaginal secretions, sweat, and spinal fluid. Such samples can control protein markers secreted from cells. Fluid samples can also be isolated from tissues isolated from a subject. For instance, the tissues can be isolated from organs including, but not limited to, brain, kidney, cartilage, lung, ovary, lymph nodes, salivary glands, breast, prostate, testes, uterus, skin and bone. A tissue sample can also be obtained from necrotic material isolated from a tumor or tumors. Such cell or group of cells may show aberrant cell growth, such as increased, uncontrolled cell proliferation and/or lack of contact inhibition. A “test fluid sample” is a fluid sample that is obtained or isolated from a subject potentially suffering from a neoplastic disease. Fluid samples potentially include a neoplastic cell or group of cells or markers from neoplastic cells. Thus, the test fluid sample can include, for example, a cancer cell that can be a hyperplastic cell, a cell from a cell line that shows a lack of contact inhibition when grown in vitro, or a cancer cell that is capable of metastasis in vivo, or a protein marker secreted or originating from a cancer cell.

As used herein, the term “test cell sample” refers to a cell, group of cells, or cells isolated from potentially cancerous tumor tissues. A test cell sample is one that potentially exhibits tumorigenic potential, metastatic potential, or aberrant growth in vivo or in vitro. A test cell sample can be isolated from any tissue, including, but not limited to, blood, bone marrow, muscle, spleen, lymph node, liver, lung, colon, thymus, kidney, brain, skin, gastrointestinal tract, eye, breast, and prostate. A test cell sample includes the cytoplasmic fraction of a cell in the cell sample.

As used herein, the term “non-neoplastic control cell sample” refers to a cell or group of cells that is exhibiting noncancerous normal characteristics for the particular cell type from which the cell or group of cells was isolated. The control cell has the same lineage as the test cell to which it is compared. A control cell sample does not exhibit tumorigenic potential, metastatic potential, or aberrant growth in vivo or in vitro. A control cell sample can be isolated from normal tissues in a subject that is not suffering from cancer. It may not be necessary to isolate a control cell sample each time a cell sample is tested for cancer as long as the nucleic acids isolated from the normal control cell sample allow for probing against the focused microarray during the testing procedure. The control cell sample may be the cytoplasmic fraction obtained from control cells.

The level of expression of KIF20A in the potentially cancerous test cell sample or potentially cancerous test fluid sample is compared to the level of expression of KIF20A in a non-neoplastic control cell or control fluid sample of the same tissue type or lineage. If the expression of KIF20A in the potentially cancerous cell or fluid sample is greater than the expression of KIF20A in the non-neoplastic control cell or fluid sample, then cancer is indicated. In some embodiments, the test cell or fluid sample is tumorigenic if the level of expression of KIF20A in the potentially cancerous cell or fluid sample is at least 1.5 times greater, at least 2 times greater, at least 4 times greater, at least 6 times greater, at least 8 times greater, at least 12 times greater, at least 15 times greater, or at least 20 times greater, than the level of expression of KIF20A in the non-neoplastic control cell or non-neoplastic fluid sample.

In embodiments in which test tissue and cell samples are used, cell samples can be isolated from human tumor tissues using means that are known in the art (see, e.g., Vara et al. (2005) Biomaterials 26(18):3987-93; Tyer et al. (1998) J. Biol. Chem. 273(5):2692-7). For example, the cell sample can be isolated from the ovary of a human patient with ovarian cancer. Other cancer cells that can be obtained include, but are not limited to, prostate cancer cells, melanoma cancer cells, osteosarcoma cancer cells, glioma cells, colon cancer cells, lung cancer cells, breast cancer cells, colon cancer and colorectal cancer cells, and leukemia cells. Cancer cells can metastasize to distant locations in the body. Non-limiting sites of metastases can include, but are not limited to, ovarian, bone, blood, lung, skin, brain, adipose tissue, muscle, gastrointestinal tissues, hepatic tissues, and kidney. Alternatively, the cell test or control cell sample can be obtained from a cell line. Cell lines can be obtained commercially from various sources (e.g., American Type Culture Collections, Mannassas, Va.). Alternatively, cell lines can be produced using techniques well known in the art.

In addition, the cell sample can be a cell line. Cancer cell lines can be created by one with skill in the art and are also available from common sources, such as the ATCC cell biology collections (American Type Culture Collections, Mannassas, Va.).

The present invention allows for the detection of cancer in tissues that are of mixed cellular populations such as a mixture of cancer cells and normal cells. In such cases, cancer cells can represent as little as 40% of the tissue isolated for the present invention to determine that the cell sample is tumorigenic. For example, the cell sample can be composed of 50% cancer cells for the present invention to detect tumorigenic potential. Cell samples composed of greater than 50% tumorigenic cells can also be used in the present invention. It should be noted that cell samples can be isolated from tissues that are less than 40% tumorigenic cells as long as the cell sample contains a portion of cells that are at least 40% tumorigenic.

Another aspect of the invention provides a method of diagnosing cancer in a fluid sample. In this method, expression of KIF20A in the fluid sample is measured. Expression levels for KIF20A can be determined using any techniques known in the art. Useful ways to determine such expression levels include, but not limited to, Western blot, protein microarrays, dipstick assays, dot blots, and Enzyme-Linked Immunosorbent Assays (“ELISA”) (see, e.g., U.S. Pat. Nos. 6,955,896, 6,087,012, 3,791,932, 3,850,752, and 4,034,074). Such examples are not intended to limit the potential means for determining the expression of a protein marker in a cell sample. Expression levels of markers in or by potentially cancerous cell samples and normal control cell samples can be compared using standard statistical techniques known to those of skill in the art (see, e.g., Ma et al. (2002) Meth. Mol. Biol. 196:139-45).

The fluid sample can be isolated from a human patient by a physician and tested for expression of KIF20A using a dipstick or any other method that relies on a solid support, solid state binding, change in color, or electric current. In addition, the cancer cell sample can be isolated from an organism that develops a tumor or cancer cells including, but not limited to, mouse, rat, horse, pig, guinea pig, or chinchilla. Cell samples can be stored for extended periods prior to testing or tested immediately upon isolation of the cell sample from the subject. Cell samples can be isolated by non-limiting methods such as surgical excision, aspiration from soft tissues such as adipose tissue or lymphatic tissue, biopsy, or removed from the blood. These methods are known to those of skill in the art.

In certain embodiments, the level of expression of anti-KIF20A antibodies in a fluid sample is detected. The level of expression of anti-KIF20A antibodies in a cell sample is detected using ELISA, Western blot, and dot blot. The level of expression of anti-KIF20A antibodies can be detected using antibodies or fragments thereof, which are directed against anti-KIF20A antibodies. The level of expression of anti-KIF20A antibodies can be detected using KIF20A-specific antibody fragments (e.g., Fab, F(ab)₂, and Fv) or whole antibodies.

A normal or cancer cell sample can be isolated from a human patient by a physician and tested for expression of protein markers using a dipstick or any other method that relies on a solid support, solid state binding, change in color, or electric current. In addition, the cancer cell sample can be isolated from an organism that develops a tumor or cancer cells including, but not limited to, mammals such as mouse, rat, horse, pig, guinea pig, rabbit, or chinchilla. Cell samples can be isolated by non-limiting methods such as surgical excision, aspiration from soft tissues such as adipose tissue or lymphatic tissue, biopsy, or removed from the blood. These methods are known to those of skill in the art. Cell samples can be stored for extended periods prior to testing or tested immediately upon isolation of the cell sample from the subject.

### 1.3 Nucleic Acid Binding Agents

In another aspect, the method of detecting cancer includes detecting a level of expression of KIF20A RNA in a test sample (i.e., neoplastic test or test fluid sample) and comparing the level of expression of KIF20A RNA detected in the test sample to the level of expression of KIF20A RNA detected in the non-neoplastic control sample. If the level of expression of KIF20A RNA is greater in the test sample than in the non-neoplastic control sample, then cancer is indicated.

As used herein, “nucleic acid binding agent” means a nucleic acid capable of hybridizing with a particular target nucleic acid sequence. Nucleic acid binding agents include any structure that can hybridize with a target nucleic acid such as an mRNA. Nucleic acids can include, but are not limited to, DNA, RNA, RNA-DNA hybrids, siRNA, and aptamers. Moreover, any detectable labels can be used so long as the label does not affect the hybridizing of the nucleic acid with its targeting. Labels include, but are not limited to, fluorophores, chemical dyes, radiolabels, chemiluminescent compounds, calorimetric enzymatic reactions, chemiluminescent enzymatic reactions, magnetic compounds, and paramagnetic compounds.

Examples of KIF20A nucleic acid sequences detected in the present invention include, but are not limited to, GenBank Accession Nos. NM_{—}005733, and BC012999.

In certain embodiments, a focused microarray can be used to detect the levels of expression of KIF20A with other markers. The term “focused microarray” as used herein refers to a device that includes a solid support with capture probe(s) affixed to the surface of the solid support. In some embodiments, the focused microarray has nucleic acids attached to a solid support. Typically, the support consists of silicon, glass, nylon or metal alloy. Solid supports used for microarray production can be obtained commercially from, for example, Genetix Inc. (Boston, Mass.). Moreover, the support can be derivatized with a compound to improve nucleic acid association. Exemplary compounds that can be used to derivatize the support include aldehydes, poly-lysine, epoxy, silane containing compounds and amines. Derivatized slides can be obtained commercially from Telechem International (Sunnyvale, Calif.).

In the case of nucleic acid binding agents, nucleic acid sequences that are selected for detecting KIF20A expression may correspond to regions of low homology between genes, thereby limiting cross-hybridization to other sequences. Typically, this means that the sequences show a base-to-base identity of less than or equal to 30% with other known sequences within the organism being studied. Sequence identity determinations can be performed using the BLAST research program located at the NIH website (world wide web at ncbi.nlm.nih.gov/BLAST). Alternatively, the Needleman-Wunsch global alignment algorithm can be used to determine base homology between sequences (see Cheung et al., (2004) FEMS Immunol. Med. Micorbiol. 40(1): 1-9.). In addition, the Smith-Waterman local alignment can be used to determine a 30% or less homology between sequences (see Goddard et al., (2003) J. Vector Ecol. 28:184-9).

Expression levels for the KIF20A can be determined using techniques known in the art, such as, but not limited to, immunoblotting, quantitative RT-PCR, microarrays, RNA blotting, and two-dimensional gel-electrophoresis (see, e.g., Rehman et al. (2004) Hum. Pathol. 35(11):1385-91; Yang et al. (2004) Mol. Biol. Rep. 31(4):241-8). Such examples are not intended to limit the potential means for determining the expression of a gene marker in a breast cancer fluid sample.

Other useful nucleic acid binding agents are specific for TTK RNA, SLC7A5 RNA, TRIM59 RNA and UHRF1 RNA. These agents can be used in combination with binding agents for KIF20A to detect neoplastic disease. In particular embodiments, a plurality of RNA for TTK, SLC7A5, TRIM59 and UHRF1 are detected with KIF20A RNA in a neoplastic test fluid or cell sample. In such embodiments, the level of expression of at least one of TTK, SLC7A5, TRIM59 and KIF20A is 1.5 times greater in a test fluid or cell sample than the level of RNA expression of the same markers in a control fluid or cell sample. In other embodiments, the level of expression of at least one of TTK, SLC7A5, TRIM59 and KIF20A is 2, 4, 5, 10, or more times greater in a test fluid or cell sample than the level of expression of the same markers in a control fluid or cell sample. The nucleic acid sequences of TTK, SLC7A5, TRIM59 and KIF20A have SEQ ID NOS: 7, 8, 9, and 10, respectively.

### 1.4 Protein-Targeting Agents

Protein marker expression is used to identify tumorigenic potential. Protein markers, such as KIF20A, can be obtained by isolation from a cell sample, or a fluid sample, using any techniques available to one of ordinary skill in the art (see, e.g., Ausubel et. al., Current Protocols in Molecular Biology, Wiley and Sons, New York, N.Y., 1999). Isolation of protein markers, including KIF20A, from the potentially tumorigenic cell sample, or from a fluid sample, obtained from a patient potentially suffering or suffering from neoplastic disease, allows for the generation of target molecules, providing a means for determining the expression level of the protein markers in the potentially tumorigenic cell or fluid sample as described below. The protein markers, such as KIF20A, can be isolated from a tissue or fluid sample isolated from a human subject. KIF20A and other protein markers can be isolated from a cytoplasmic fraction or a membrane fraction of the sample. Protein isolation techniques known in the art include, but are not limited to, column chromatography, spin column chromatography, and protein precipitation. KIF20A can be isolated using methods that are taught in, for example, Ausubel et al., Current Protocols in Molecular Biology, Vol. 1, John Wiley & Sons, Inc., (1993).

The invention provides protein-targeting agents such as binding agents, e.g., antibodies or KIF20A-binding fragments thereof. These embodiments are described in detail below. Other potential protein targeting agents include, but are not limited to, transforming acidic coil-coil (TACC) (see, e.g., Don, et al. (2004) FEBS Lett. 572:51-56) aptamers, and ligands specific for KIF20A peptidomimetic compounds, peptides directed to the active sites of an enzyme, and nucleic acids.

Inhibitors can also be used as protein targeting agents to bind to protein markers. Useful inhibitors are compounds that bind to a target protein, and normally reduce the “effective activity” of the target protein in the cell or cell sample. Inhibitors include, but are not limited to, antibodies, antibody fragments, peptides, peptidomimetic compounds, and small molecules (see, e.g., Lopez-Alemany et al. (2003) Am. J. Hematol. 72(4): 234-42; Miles et al (1991) Biochem. 30(6): 1682-91). Inhibitors can perform their functions through a variety of means including, but not limited to, non-competitive, uncompetitive, and competitive mechanisms.

Protein-targeting agents can also be conjugated to non-limiting materials such as magnetic compounds, paramagnetic compounds, proteins, nucleic acids, antibody fragments, or combinations thereof. Furthermore, protein-targeting agents can be disposed on an NPV membrane and placed into a dipstick. Protein-targeting agents can also be immobilized on a solid support at pre-determined positions such as in the case of a microarray. For instance, antibodies can be printed or cross-linked via their Fc regions to pre-derivatized surfaces of solid supports. In addition, antibodies can be cross-linked using bifunctional crosslinkers to a functionalized solid support. Such bifunctional crosslinking is well known in the art (see, e.g., U.S. Pat. Nos. 7,179,447 and 7,183,373).

Crosslinking of protein-targeting agents, such as antibodies and other proteins, to a water-insoluble support matrix can be performed with bifunctional agents well known in the art including 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Bifunctional agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates can be employed for protein immobilization.

Protein-targeting agents can be detectably labeled. As used herein, “detectably labeled” means that a targeting agent is operably linked to a moiety that is detectable. By “operably linked” is meant that the moiety is attached to the protein-targeting agent by either a covalent or non-covalent (e.g., ionic) bond. Methods for creating covalent bonds are known (see, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y., 1999).

According to the invention, a “detectable label” is a moiety that can be sensed. Such labels can be, without limitation, fluorophores (e.g., fluorescein (FITC), phycoerythrin, rhodamine), chemical dyes, or compounds that are radioactive, chemiluminescent, magnetic, paramagnetic, promagnetic, or enzymes that yield a product that may be colored, chemiluminescent, or magnetic. The signal is detectable by any suitable means, including spectroscopic, photochemical, biochemical, immunochemical, electrical, optical or chemical means. In certain cases, the signal is detectable by two or more means. In certain embodiments, protein targeting agents include fluorescent dyes, radiolabels, and chemiluminescent labels, which are examples that are not intended to limit the scope of the invention (see, e.g., Gruber et al. (2000) Bioconjug. Chem. 11(5): 696-704).

For example, protein-targeting agents may be conjugated to Cy5/Cy3 fluorescent dyes. These dyes are frequently used in the art (see, e.g., Gruber et al. (2000) Bioconjug. Chem. 11(5): 696-704). The fluorescent labels can be selected from a variety of structural classes, including the non-limiting examples such as 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein).

Aspects of the present invention utilize monoclonal and polyclonal antibodies as protein targeting agents directed specifically against certain cancer marker proteins, particularly KIF20A. Anti-KIF20A protein antibodies, both monoclonal and polyclonal, for use in the invention are available from several commercial sources (e.g., Bethyl laboratories, Lifespan Biosciences, Abcam, Abnova (Cederlane)). Other useful markers to which protein targeting agents such as antibodies can be provided include, but are not limited to, UHRF1, TTK, SLC7A5, and TRIM59. UHRF1, TTK, SLC7A5, TRIM59 and KIF20A antibodies can be administered to a patient orally, subcutaneously, intramuscularly, intravenously, or interperitoneally for in vivo detection and/or imaging. In certain embodiments, KIF20A is used alone as a protein marker to diagnose cancer.

Aspects of the invention also utilize polyclonal antibodies for the detection of KIF20A, TTK, SLC7A5, TRIM59 and KIF20A. They can be prepared by known methods or commercially obtained.

As used herein, the term “polyclonal antibodies” means a population of antibodies that can bind to multiple epitopes on an antigenic molecule. A polyclonal antibody is specific to a particular epitope on an antigen, while the entire group of polyclonal antibodies can recognize different epitopes. In addition, polyclonal antibodies developed against the same antigen can recognize the same epitope on an antigen, but with varying degrees of specificity. Polyclonal antibodies can be isolated from multiple organisms including, but not limited to, rabbit, goat, horse, mouse, rat, and primates. Polyclonal antibodies can also be purified from crude serums using techniques known in the art (see, e.g., Ausubel, et al., Current Protocols in Molecular Biology, Vol. 1, pp. 4.2.1-4.2.9, John Wiley & Sons, Inc., 1996).

The term “monoclonal antibody”, as used herein, refers to an antibody obtained from a population of substantially homogenous antibodies, i.e., the individual antibodies comprising the population are identical except for possible naturally occurring mutations that may be present in minor amounts. Monoclonal antibodies are highly specific, being directed against a single antigenic site. By their nature, monoclonal antibody preparations are directed to a single specific determinant on the target. Novel monoclonal antibodies or fragments thereof mean in principle all immunoglobulin classes such as IgM, IgG, IgD, IgE, IgA, or their subclasses or mixtures thereof. Non-limiting examples of subclasses include the IgG subclasses IgG1, IgG2, IgG3, IgG2a, IgG2b, IgG3, or IgGM. The IgG subtypes IgG1/κ and IgG2b/κ are also included within the scope of the present invention. Antibodies can be obtained commercially from, e.g., BioMol International LP (Plymouth Meeting, Pa.), BD Biosciences Pharmingen (San Diego, Calif.), and Cell Sciences, Inc. (Canton, Mass.).

The monoclonal antibodies herein include hybrid and recombinant antibodies produced by splicing a variable (including hypervariable) domain of an anti-biomarker protein antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567; Mage and Lamoyi, in Monoclonal Antibody Production Techniques and Applications, (Marcel Dekker, Inc., New York 1987, pp. 79-97). Thus, the modified “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method (see, e.g., Kohler and Milstein (1975) Nature 256:495) or can be made by recombinant DNA methods (U.S. Pat. No. 4,816,567). The monoclonal antibodies can also be isolated from phage libraries generated using the techniques described in the art (see, e.g., McCafferty et al (1990) Nature 348:552-554).

Alternative methods for producing antibodies can be used to obtain high affinity antibodies. Antibodies can be obtained from human sources such as serum. Additionally, monoclonal antibodies can be obtained from mouse-human heteromyeloma cell lines by techniques known in the art (see, e.g., Kozbor (1984) J. Immunol. 133, 3001; Boerner et al., (1991) J. Immunol. 147:86-95). Methods for the generation of human monoclonal antibodies using phage display, transgenic mouse technologies, and in vitro display technologies are known in the art and have been described previously (see, e.g., Osbourn et al. (2003) Drug Discov. Today 8: 845-51; Maynard and Georgiou (2000) Ann. Rev. Biomed. Eng. 2: 339-76; U.S. Pat. Nos. 4,833,077, 5,811,524, 5,958,765, 6,413,771, and 6,537,809).

In addition, aptamers can be protein targeting agents. The term “aptamer,” used herein interchangeably with the term “nucleic acid ligand,” means a nucleic acid that, through its ability to adopt a specific three-dimensional conformation, binds to and has an antagonizing (i.e., inhibitory) effect on a target. The target of the present invention is KIF20A, and hence the term KIF20A aptamer or “KIF20A nucleic acid ligand” is used. Aptamers may be made to other biomarkers as well. The aptamer can bind to the target by reacting with the target, by covalently attaching to the target, or by facilitating the reaction between the target and another molecule. Aptamers may be comprised of multiple ribonucleotide units, deoxyribonucleotide units, or a mixture of both types of nucleotide residues. Aptamers may further comprise one or more modified bases, sugars or phosphate backbone units as described above.

Aptamers can be made by any known method of producing oligomers or oligonucleotides. Many synthesis methods are known in the art. For example, 2′-O-allyl modified oligomers that contain residual purine ribonucleotides, and bearing a suitable 3′-terminus such as an inverted thymidine residue (Ortigao et al., (1992) Antisense Res. Devel. 2:129-146) or two phosphorothioate linkages at the 3′-terminus to prevent eventual degradation by 3′-exonucleases, can be synthesized by solid phase beta-cyanoethyl phosphoramidite chemistry (Sinha et al., Nucleic Acids Res., 12:4539-4557 (1984)) on any commercially available DNA/RNA synthesizer. Purification can be performed either by denaturing polyacrylamide gel electrophoresis or by a combination of ion-exchange HPLC (Sproat et al., (1995) Nucleosides and Nucleotides, 14:255-273) and reversed phase HPLC. For use in cells, synthesized oligomers are converted to their sodium salts by precipitation with sodium perchlorate in acetone. Traces of residual salts may then be removed using small disposable gel filtration columns that are commercially available. As a final step the authenticity of the isolated oligomers may be checked by matrix assisted laser desorption mass spectrometry (Pieles et al., (1993) Nucleic Acids Res., 21:3191-3196) and by nucleoside base composition analysis.

There are several techniques that can be adapted for refinement or strengthening of the nucleic acid ligands binding to a particular target molecule or the selection of additional aptamers. One technique has been termed Selective Evolution of Ligands by Exponential Enrichment (SELEX). Compositions and methods for generating aptamer antagonists of the invention by SELEX and related methods are known in the art and taught in, for example, U.S. Pat. Nos. 5,475,096 and 5,270,163. The SELEX process in general is further described in, e.g., U.S. Pat. Nos. 5,668,264, 5,696,249, 5,670,637, 5,674,685, 5,723,594, 5,756,291, 5,811,533, 5,817,785, 5,958,691, 6,011,020, 6,051,698, 6,147,204, 6,168,778, 6,207,816, 6,229,002, 6,426,335, and 6,582,918.

The detection of cancer markers can also be accomplished using protein microarrays. Protein microarrays can be prepared by methods disclosed in, e.g., U.S. Pat. Nos. 6,087,102, 6,139,831, and 6,087,103. In addition, protein-targeting agents conjugated to the surface of the protein microarray can be bound by detectably labeled protein markers isolated from a cell sample or a fluid sample. This method of detection can be termed “direct labeling” because the protein marker, which is the target, is labeled. In other embodiments, protein markers can be bound by protein-targeting agents, and then subsequently bound by a detectably labeled antibody specific for the protein marker. These methods are termed “indirect labeling” because the detectable label is associated with a secondary antibody or other protein-targeting agent. An overview of protein microarray technology in general can be found in Mitchell, Nature Biotech. (2002), 20:225-229, the contents of which are incorporated herein by reference.

### 1.5 Detection of KIF20A and Other Markers in Biological Fluids

An aspect of the present invention includes an assay for the detection of KIF20A and other cancer markers in biological fluid samples using a protein-targeting agent to bind to the KIF20A protein. Protein-targeting agents can bind to KIF20A protein that is obtained from tissue or biological fluids.

As used herein, the term “biological fluids” means aqueous or semi-aqueous liquids isolated from an organism in which biological macromolecules may be identified or isolated. Biological fluids may be disposed internally as in the case of blood, serum, amniotic fluid, bile, or cerebrospinal fluid. Biological fluids can be excreted as in the non-limiting cases of urine, saliva, sweat, mucosal secretions, vaginal secretions, lacrimal secretions, seminal fluid, seminal fluid, and sebaceous secretions.

For detection of markers in biological fluids, detection devices can be used that are in the form of a “dipstick.” Such devices are known in the art, and have been applied to detecting KIF20A protein in serum and other biological fluids (see, e.g. U.S. Pat. No. 4,390,343). In some instances, a dipstick-type device can be comprised of analytical elements where protein-targeting agents, such as antibodies, inhibitors, organic molecules, peptidomimetic compounds, ligands, organic compounds, or combinations thereof, are incorporated into a gel. The gel can be comprised of non-limiting substances such as agarose, gelatin or PVP (see, e.g., U.S. Pat. No. 4,390,343). The gel can be contained within an analytical region for reaction with a protein marker.

The “dipstick” format (exemplified in U.S. Pat. Nos. 5,275,785, 5,504,013, 5,602,040, 5,622,871 and 5,656,503) typically consists of a strip of porous material having a biological fluid sample-receiving end, a reagent zone and a reaction zone. As used herein, the term “reagent zone” means the area within the dipstick in which the protein-targeting agent and the KIF20A protein in the biological sample come into contact. By the term “reaction zone”, is meant the area within the dipstick in which an immobilized binding agent captures the protein-targeting agent/protein marker complex. As used herein, the term “binding agent” refers to any molecule or group of molecules that can bind, interact, or associate with a protein-targeting agent/protein marker complex.

In certain embodiments, the biological fluid sample is wicked along the assay device starting at the sample-receiving end and moving into the reagent zone. The protein marker(s) to be detected binds to a protein-targeting agent incorporated into the reagent zone, such as a labeled protein-targeting agent, to form a complex. For example, a labeled antibody can be the protein-targeting agent, which complexes specifically with the protein marker. In other examples, the protein-targeting agent can be a receptor that binds to a protein marker in a receptor:ligand complex. In yet other examples, an inhibitor is used to bind to a protein marker, thereby forming a complex with the protein marker targeted by the particular inhibitor. In some examples, peptidomimetic compounds are used to bind to KIF20A protein to mimic the interaction of a protein marker with a normal peptide. In other examples, the protein-targeting agent can be an organic molecule capable of associating with the protein marker. In all cases, the protein-targeting agent has a label. The labeled protein-targeting agent-protein marker complex then migrates into the reaction zone, where the complex is captured by another specific binding partner firmly immobilized in the reaction zone. Retention of the labeled complex within the reaction zone thus results in a visible readout.

A number of different types of other useful assays that measure the presence of a protein market are well known in the art. One such assay is an immunoassay. Immunoassays may be homogeneous, i.e. performed in a single phase, or heterogeneous, where antigen or antibody is linked to an insoluble solid support upon which the assay is performed. Sandwich or competitive assays may be performed. The reaction steps may be performed simultaneously or sequentially. Threshold assays may be performed, where a predetermined amount of analyte is removed from the sample using a capture reagent before the assay is performed, and only analyte levels of above the specified concentration are detected. Assay formats include, but are not limited to, for example, assays performed in test tubes, wells or on immunochromatographic test strips, as well as dipstick, lateral flow or migratory format immunoassays.

A lateral flow test immunoassay device may be used in this aspect of the invention. In such devices, a membrane system forms a single fluid flow pathway along the test strip. The membrane system includes components that act as a solid support for immunoreactions. For example, porous or bibulous or absorbent materials can be placed on a strip such that they partially overlap, or a single material can be used, in order to conduct liquid along the strip. The membrane materials can be supported on a backing, such as a plastic backing. The test strip includes a glass fiber pad, a nitrocellulose strip and an absorbent cellulose paper strip supported on a plastic backing.

Antibodies that specifically bind with KIF20A or other target protein markers can be immobilized on the solid support. The antibodies can be bound to the test strip by adsorption, ionic binding, van der Waals adsorption, electrostatic binding, or by covalent binding, by using a coupling agent, such as glutaraldehyde. For example, the antibodies can be applied to the conjugate pad and nitrocellulose strip using standard dispensing methods, such as a syringe pump, airbrush, ceramic piston pump or drop-on-demand dispenser. A volumetric ceramic piston pump dispenser can be used to stripe antibodies that bind the analyte of interest, including a labeled antibody conjugate, onto a glass fiber conjugate pad and a nitrocellulose strip.

The test strip can be treated, for example, with sugar to facilitate mobility along the test strip or with water-soluble non-immune animal proteins, such as albumins, including bovine (BSA), other animal proteins, water-soluble polyamino acids, or casein to block non-specific binding sites.

### 1.6 Cancer Diagnosis and Prediction Analysis

Cancer diagnoses can be performed by comparing the levels of expression of a protein marker, such as KIF20A, or a set of protein markers including KIF20A in a potentially neoplastic cell sample to the levels of expression for a protein marker or a set of protein markers in a normal control cell sample of the same tissue type. Alternatively, the level of expression of a protein marker, such as KIF20A, or a set of protein markers in a potentially cancerous cell sample is compared to a reference group of protein markers that represents the level of expression for a protein marker or a set of protein markers in a normal control population (herein termed “training set”). The training set also includes the data for a population that has a known tumor or class of tumors. This data represents the average level of expression that has been determined for the neoplastic cells isolated from the tumor or class of tumors. It also has data related to the average level of expression for a protein marker or set of protein markers for normal cells of the same cell type within a population. In these embodiments, the algorithm compares newly generated expression data for a particular protein marker or set of protein markers from a cell sample isolated from a patient containing potentially neoplastic cells to the levels of expression for the same protein marker or set of protein markers in the training set. The algorithm determines whether a cell sample is neoplastic or normal by aligning the level of expression for a protein marker or set of protein markers with the appropriate group in the training set. In certain embodiments, software for performing the statistical manipulations described herein can be provided on a computer connected by data link to a data generating device, such as a microarray reader.

Class prediction algorithms can be utilized to differentiate between the levels of expression of markers in a cell sample and the levels of expression of markers in a normal cell sample (Vapnik, The Nature of Statistical Learning Theory, Springer Publishing, 1995). Exemplary, non-limiting algorithms include, but are not limited to, compound covariate predictor, diagonal linear discriminant analysis, nearest neighbor predictor, nearest centroid predictor, and support vector machine predictor (Simon et al., Design and Analysis of DNA Microarray Investigations: An Artificial Intelligence Milestone., Springer Publishing, 2003). These statistical tests are well known in the art, and can be applied to ELISA or data generated using other protein expression determination techniques such as dot blotting, Western blotting, and protein microarrays (see, e.g., U.S. Pub. No. 2005/0239079).

It should be recognized that statistical analysis of the levels of expression of protein markers in a cell sample to determine cancer state does not require a particular algorithm or set of particular algorithms. Any algorithm can be used in the present invention so long as it can discriminate between statistically significant and statistically insignificant differences in the levels of expression of protein markers in a cell sample as compared to the levels of expression of the same protein markers in a normal cell sample of the same tissue type. In this case, a test sample is considered cancerous or malignant if the expression of one or more protein marker is above a cut-off value established for one or all markers in normal or control samples.

In some embodiments, an increased level of expression in the potentially cancerous cell sample, or fluid sample, indicates that cancer cells exist in the cell sample. In such cancerous samples, protein markers showing increased levels of expression include, but are not limited to, KIF20A, as well as TTK, SLC7A5, TRIM59 and UHRF1. The algorithm makes the class prediction based upon the overall levels of expression found in the cell sample as compared to the levels of expression in the training set. It should be noted that, in some instances, KIF20A can be used to classify a sample as either neoplastic or normal. Two, three, four, five, six, or more protein markers, including KIF20A, can also be used to properly classify a cell sample as neoplastic or normal. In particular, three protein markers, including KIF20A, can be used for classification purposes. Four protein markers, including KIF20A, can be used to identify neoplastic cells within a cell sample. Five protein markers, including KIF20A, can be used to identify neoplastic cells in a cell sample. Furthermore, six or more protein markers, including KIF20A, can be used to properly classify cell samples into either the neoplastic cell class or the non-neoplastic cell class.

The type of analysis detailed above compares the level of expression for the protein marker(s) in the cell sample to a training set containing reference groups of protein that are representative of a normal population and a neoplastic population. In certain embodiments, the training set can be obtained with kits that can be used to determine the level of expression of protein marker(s) in a patient cell sample. Alternatively, an investigator can generate new training sets using protein expression reference groups that can be obtained from commercial sources such as Asterand, Inc. (Detroit, Mich.). Comparisons between the training sets and the cell samples are performed using standard statistical techniques that are well known in the art, and include, but are not limited to, the ArrayStat 1.0 program (Imaging Research, Inc., Brock University, St. Catherine's, Ontario, Calif.). Statistically significant increased levels of expression in the cell sample of protein marker(s) indicate that the cell sample contains a cancer cell or cells with tumorigenic potential. Also, standard statistical techniques such as the Student T test are well known in the art, and can be used to determine statistically significant differences in the levels of expression for protein markers in a patient cell sample (see, e.g., Piedra et al. (1996) Ped. Infect. Dis. J. 15: 1). In particular, the Student T test is used to identify statistically significant changes in expression using protein microarray analysis or ELISA analysis (see, e.g., Piedra et al. (1996) Ped. Infect. Dis. J. 15:1).

### 1.7 Kits

Aspects of the invention additionally provide kits for detecting neoplasms such as ovarian, lung, breast, colon and prostate cancers in a cell or a fluid sample. The kits include targeting agents for the detection of KIF20A, or KIF20A and at least one of biomarkers TTK, SLC7A5, TRIM59 and/or UHRF1. In certain embodiments, kits include targeting agents for the detection of KIF20A. A patient that potentially has a tumor or the potential to develop a tumor (“in need thereof”) can be tested for the presence of a tumor or tumor potential by determining the level of expression of targeting agents in a cell or fluid sample derived from the patient.

The kit comprises labeled binding agents capable of detecting KIF20A, or KIF20A and at least one of UHRF1, TTK, SLC7A5, TRIM59 and/or KIF20A in a biological sample, as well as means for determining the amount of these protein markers in the sample, and means for comparing the amount of the protein markers in the potentially cancerous sample with a standard (e.g., normal non-neoplastic control cells). The binding agents can be packaged in a suitable container. The kit can further comprise instructions for using the compounds or agents to detect the protein markers, as well as other neoplasm-associated markers. Such a kit can comprise, e.g., one or more antibodies, or biomarker-specific binding fragments thereof as binding agents, that bind specifically to at least a portion of a protein marker.

In particular, kits comprise labeled binding agents capable of binding to and detecting KIF20A, as well as means determining the amount of KIF20A in the sample, and means for comparing the amount of the protein markers in the potentially cancerous sample with a standard (e.g., normal non-neoplastic control cells). Such a kit can comprise, e.g., one or more antibodies, or biomarker-specific binding fragments thereof as binding agents, that bind specifically to at least a portion of a KIF20A.

The kit can also contain a probe for detection of housekeeping protein expression. These probes advantageously allow health care professionals to obtain an additional data point to determine whether a specific or general cancer treatment is working so KIF20A levels can be used to monitor the success of cancer treatment, and are used to normalize the signal obtained between patients. As used herein, the term “housekeeping proteins” refers to any protein that has relatively stable or steady expression at the protein level during the life of a cell. Housekeeping proteins can be protein markers that show little difference in expression between cancer cells and normal cells in a particular tissue type. Examples of housekeeping proteins are well known in the art, and include, but are not limited to, isocitrate lyase, acyltransferase, creatine kinase, TATA-binding protein, hypoxanthine phosphoribosyl transferase 1, and guanine nucleotide binding protein, beta polypeptide 2-like 1 (see, e.g., Pandey et al. (2004) Bioinformatics 20(17): 2904-2910). In addition, the housekeeping proteins are used to identify the proper signal level by which to compare the cell sample signals between proteins from different or independent experiments. The probes can be any binding agents such as labeled antibodies, or fragments thereof, specific for the housekeeping proteins. Alternatively or additionally, the probes can be inhibitors, peptidomimetic compounds, peptides and/or small molecules.

Data related to the levels of expression of the selected protein markers in normal tissues and neoplasms can be supplied in a kit or individually in the form of a pamphlet, document, floppy disk, or computer CD. The data can represent patient groups developed for a particular population (e.g., Caucasian, Asian, etc.) and is tailored to a particular cancer type. Such data can be distributed to clinicians for testing patients for the presence of a neoplasm. A clinician obtains the levels of expression for a protein marker or set of protein markers in a particular patient. The clinician then compares the expression information obtained from the patient to the levels of expression for the same protein marker or set of protein markers that had been determined previously for both normal control and cancer patient groups. A finding that the level of expression for the protein marker or the set of protein markers is similar to the normal patient group data indicates that the cell sample obtained from the patient is not neoplastic. A finding that the level of expression for the protein marker or the set of protein markers is similar to the cancer patient group data indicates that the cell sample obtained from the patient is neoplastic

### 1.8 Testing

The diagnostic methods according to the invention were tested for their ability to diagnose cancer in test cell samples isolated from human subjects suffering from ovarian cancer, lung cancer, prostate cancer, hepatic cancer, pancreatic cancer, breast cancer, leukemia, sarcoma, melanoma, renal cancer, colon cancer, and osteosarchma.

The expression levels of KIF20A RNA and KIF20A protein in combination with other cancer markers were analyzed for differential expression in lung, breast, ovarian, colon and prostate samples by Real-time PCR and Western blot. The testing and results are described in detail below in the Examples.

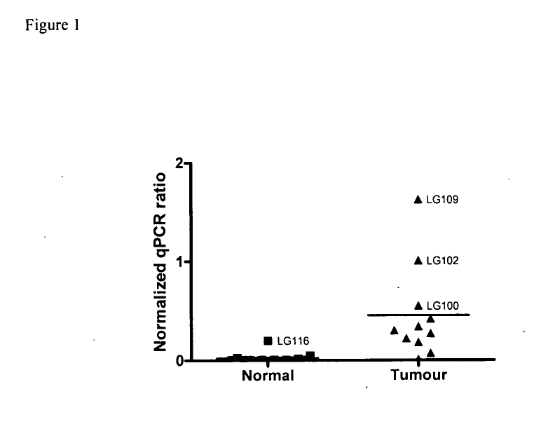

FIG. 1 shows that KIF20A RNA expression is increased in lung tumor tissues as compared to normal lung tissues. These results indicate that the increase in KIF20A expression is a marker of the transformation of normal lung cells to neoplastic lung cells.

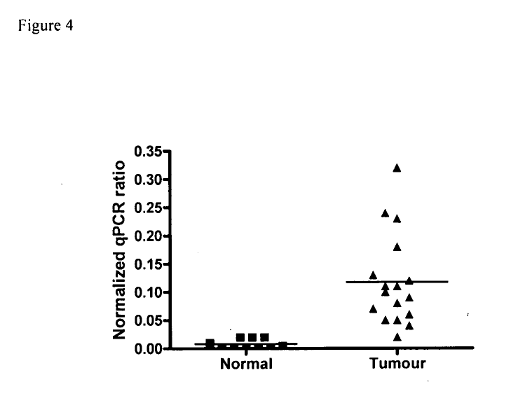

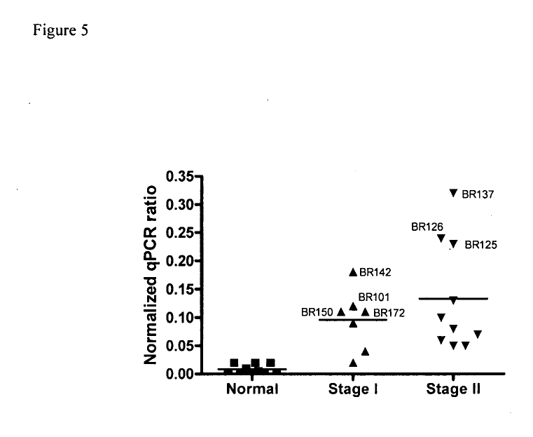

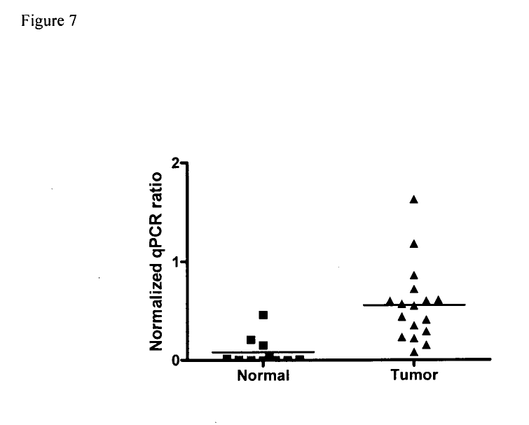

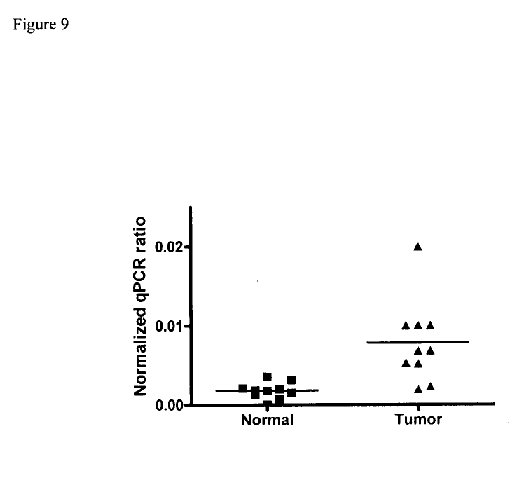

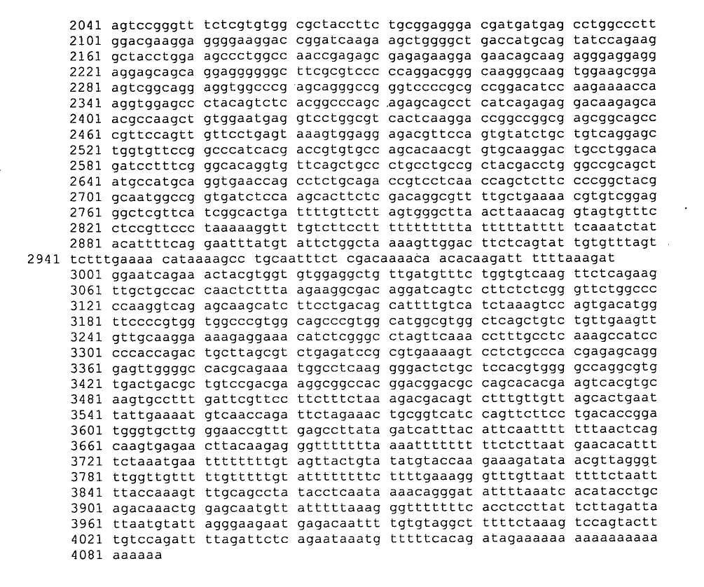

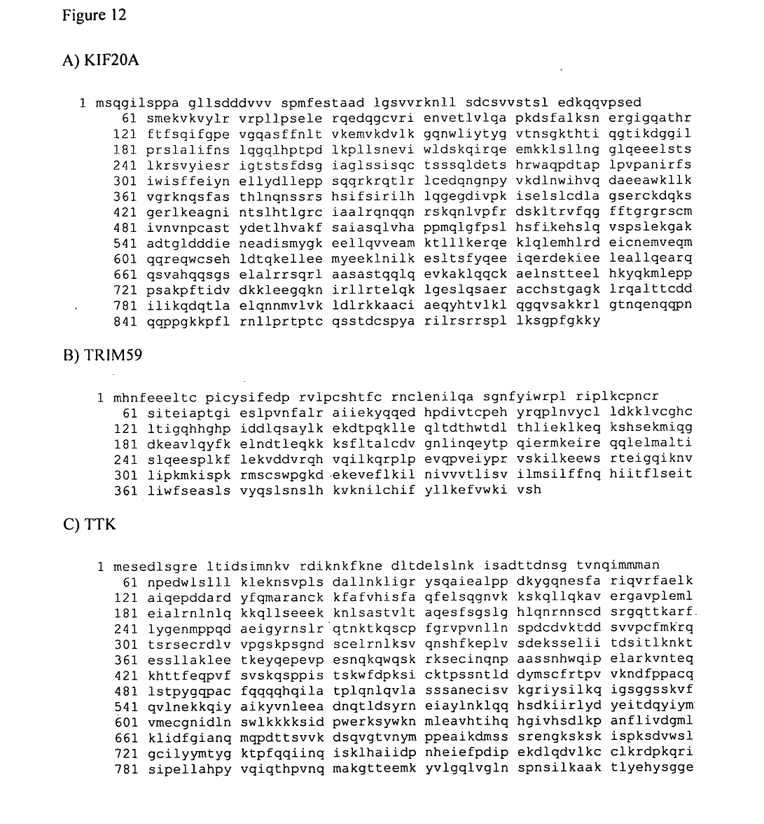

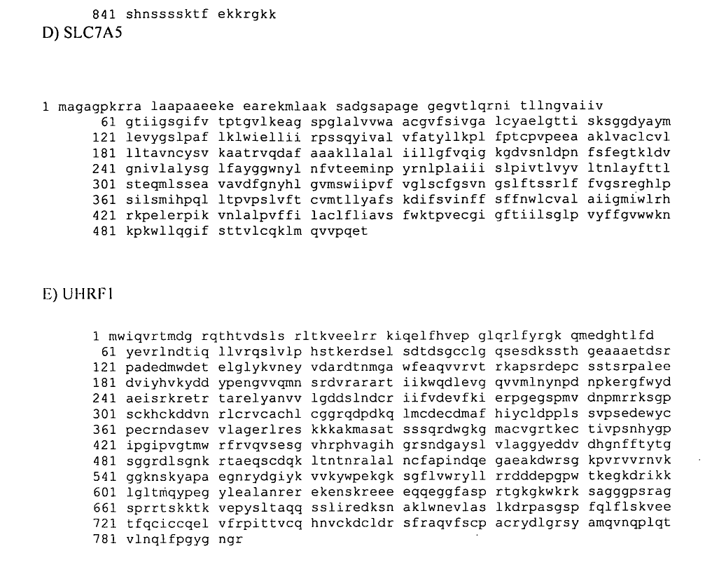

Increased expression of KIF20A RNA was also observed in breast cancer patient samples as compared to normal tissue samples (FIGS. 4-5). In addition, ovarian cancer samples showed higher levels of RNA expression as compared to normal ovarian tissues (FIG. 7). Similarly, KIF20A RNA expression was increased in colorectal cancer samples versus normal colon tissues from patients (FIG. 9). However, KIF20A RNA expression did not show a significant increase in prostate cancer samples when compared to normal prostate tissue samples (FIG. 11).

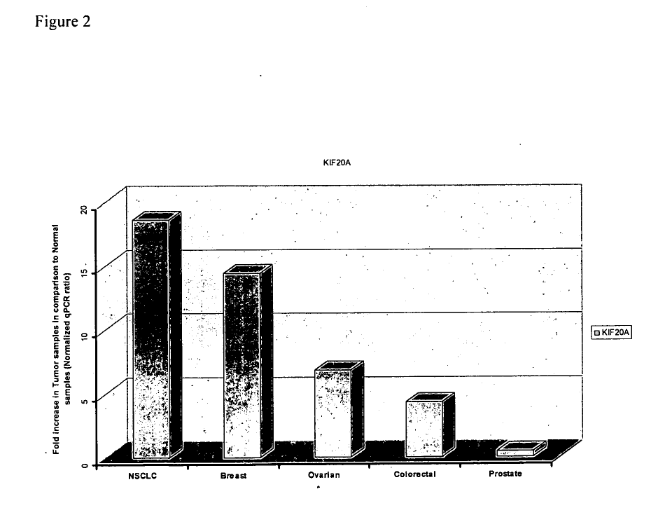

FIG. 2 and Table 1 summarize the results of the RNA experiments by showing the normalized Real-time PCR ratios of KIF20A expression levels found in lung (NSCLC), breast, ovarian, colorectal and Stage I prostate cancer patients and normal tissue subjects. In summary, RNA expression lung, breast, ovarian, and colon studies show that KIF20A is a marker of the transformation of normal cells to neoplastic cells of the same lineage.

TABLE 1

| Cancer type | KIF20A |
|---|---|
| NSCLC | 18.6 |
| Breast | 14.4 |
| Ovarian | 6.8 |
| Colorectal | 4.3 |
| Prostate | 0.43 |

Table 2 shows a compilation of KIF20A expression results in cell lines from various cancers as compared with tissue mached controls.

TABLE 2

| | | KIF20A |
|---|---|---|
| | | expression |
| Cancer type | Cell lines | level |
| Breast | MCF7 | 157.69 |
| | MDA | 2777.79 |
| Ovarian | SKOV3 | 14.34 |
| | 2008 | 19.23 |
| | OVCAR-3 | 37.73 |
| Colorectal | T84 | 38.12 |
| | HCT116 | 6.13 |
| Lung | H460 | 214.98 |
| | A549 | 15.35 |
| Prostate | PC3 | 271.52 |

Other markers were also tested for differential expression in lung, breast, ovarian, colorectal and prostate tissues. There is a significant increase in TTK, SLC7A5, TRIM59 and KIF20A RNA expression in lung (NSCLC) cancer versus normal lung tissues. Similar increase in RNA expression of TTK, SLC7A5, TRIM59 and KIF20A is seen in breast, ovarian, and colorectal cancers versus normal tissues for each respective cancer. These results indicate that these proteins can be used as markers of certain neoplastic disease in combination with KIF20A.

Table 3 shows a compilation of the RNA expression results found in lung, breast, ovarian, and colorectal cancer tissues as compared to tissue-matched controls, together with the quantified fold increases for TTK, SLC7A5, TRIM59, UHRF1 and KIF20A RNAs.

TABLE 3

| | Breast | | Ovarian | | | Colon | | Lung | |
|---|---|---|---|---|---|---|---|---|---|
| ABP Biomarkers | MCF7 | MDA | SKOV3 | 2008 | OVCAR3 | T84 | HCT116 | H460 | A549 |
| TTK | 157.7 | 2777.8 | 14.3 | 19.2 | 37.7 | 38.1 | 6.1 | 215 | 15.4 |
| SLC | 75.4 | 8.7 | 19.2 | 57.9 | 8.6 | 25.2 | 96.1 | 169 | 56.8 |
| TRIM59 | 6.9 | 9.5 | 12.7 | 8.6 | 28.9 | 11.8 | 9.2 | 8 | 45.1 |
| KIF20A | 18.7 | 36.7 | 11.3 | 5.6 | 16.2 | 19.2 | 9.1 | 53.9 | 29.1 |
| UHRF1 | 8.6 | 30.5 | 8.7 | 5.6 | 5.2 | 8.8 | 8.4 | 74.9 | 68.5 |

In all, these results, in combination with the results described in the examples, indicate that KIF20A alone, or in combination with TRIM59, TTK, SLC7A5 and/or UHRF1 described herein, is a marker of certain neoplastic diseases.

### EXAMPLES

Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Human Genome Oligoarray Experiments

### 1. Tumor Cell Lines

Human breast adenocarcinoma cell line MCF7, human ovarian adenocarcinoma cell line SKOV3, human ovarian carcinoma cell line 2008, human colorectal carcinoma cell lines T84 and HCT116, human lung carcinoma cell line A549, human non-small cell lung carcinoma cell line NCI-H460 and human prostatic adenocarcinoma cell line PC3 were obtained from ATCC (Manassas, Va., USA). All cell culture materials were obtained from Gibco Life Technologies (Burlington, Ont., Canada). The cell lines were cultured in αMEM medium supplemented with 10% FBS (MCF7) or with 15% FBS (SKOV3), in RPMI 1640 medium supplemented with 5% FBS (T84) or 10% FBS (H460) or 15% FBS (2008) or 20% FBS and 2.5% Glucose, 0.1 M HEPES, 10 mM MEM sodium pyruvate and 10 μmol/ml bovine insuline (OVCAR-3), in Dulbecco's Modified Eagle Medium supplemented with 10% FBS (MDA), in McCoy's 5A Medium Modified supplemented with 10% FBS (HCT116), in HAM's F12 Medium supplemented with 10% FBS (A549, PC3). All culture media contained L-glutamine (final concentration of 2 mM). The cells were grown in the absence of antibiotics at 37° C. in a humid atmosphere of 5% CO₂ and 95% air. All cell lines were determined to be free of mycoplasma contamination using a PCR-based mycoplasma detection kit according to manufacturer's instructions commercially available (Stratagene Inc., San Diego, Calif., USA).

### 2. Cell RNA Extraction

Total RNA extraction from cell lines was done with RNEasy kit (Qiagen, USA), following the manufacturer's recommendations. Quantification of the RNA is done with the Nanodrop® ND-1000 spectrophotometer and the quality is assessed by the A₂₆₀/A₂₈₀ ratio. RNA preparations with an absorpance (A₂₆₀/A₂₈₀ ratio) of 1.9 to 2.3 were used for gene profiling experiments.

### 3. Normal Total RNA Groups

Total RNA groups for breast, ovarian, colon, lung and prostate were purchased from Biochain Institute Inc. (Hayward, USA). Standard clinical data were available for each patient included in the groups. Total RNA was extracted from snap frozen tissues samples using Trizol Reagent kit (Gibco-BRL, USA) extraction procedure. Total RNA was treated with RNA-free DNAse I and purified with the RNEasy kit (Qiagen, USA). RNA samples were visualized and analyzed on an Agilent 2100 BioAnalyzer (Agilent, USA) for purity and integrity.

### 4. Transcriptional Profiling

Fluorescently labeled cDNAs were prepared from 20 μg of total RNA for cancerous cell line and the normal human total RNA groups using the Agilent Fluorescent Direct Label kit (Agilent Technologies) using 1.0 mM cyanine 3- or 5-labeled dCTPs (Perkin Elmer, Waltham, Mass.) according to the manufacturer's instructions. cDNA preparations from tumor cell linnes were cyanine-labeled and mixed with the reverse-color-labeled cDNA prepared from normal human total RNA group. Hybridizations were performed using the Agilent in situ Hybridization Plus kit according to the manufacturer's recommendations (Agilent Technologies). The combined cyanine 3- and 5-labeled cDNAs were denatured at 98° C. for 3 min, cooled to RT, and complemented with 50 μl of 10× control targets and 250 μl of 2× hybridization buffer. The labeled material was then applied to the Agilent Whole human genome oligo microarray (Agilent Technologies, #G4112A) consisting of 44,000 known and unknown human genes printed as 60-mer oligonucleotides using the SurePrint technology. The microarrays were hybridized in a hybridization rotation oven at 60° C. for 15 hr. The slides were disassembled in 6×SSC+0.005% Triton X-102, and washed with 6×SSC+0.005% Triton X-100 for 10 min at RT, followed by 5 min at 4° C. in 0.1×SSC+0.005% Triton X-100. Lastly, the slides were spun dry for 5 min at 1000 rpm. The microarrays were scanned with the ScanArray Lite scanner (Perkin Elmer), and the raw image data were extracted with the Packard BioScience QuantArray® Microarray Analysis software. Data were analyzed with the ImaGene v6.0 software (BioDiscovery Inc, El Segundo, Calif.).

### 5. Microarray Data Analysis

The ImaGene® 6.0 was used to generate the lists of differentially expressed genes for each experiment. First, automated spot flagging analysis schemes were used to remove suspicious spots from any further analysis. Then, local methods for background correction measurement were applied. A log 10 transformation was done on the background-corrected data, followed by a global Lowess normalization step (based on intensity-dependent values) with a smoothing factor of 0.2. Finally, the background-corrected and normalized signals were analyzed to generate up and down regulated genes lists with a fold change threshold of 2.0. Moreover, a dye swap reaction was performed for one resistant/sensitive cell line (on the same day to account for potential differential incorporation of the labeled dCTPs used in the cDNA labeling reactions). Data analysis indicated that direct and reverse experiments performed with the same total RNA preparation gave similar gene profiling patterns, regardless of the date experiments were performed. When compared the greater than 10-fold up-regulated genes between the two experiments (direct and dye swap), 96% of them were the same. As for the down-regulated genes, 93% of them were the same in both experiments. Therefore, the tumor markers were selected based of the expression profiling done on the direct labeling experiment for the each of the cell lines tested.

Filtered- and Lowess-normalized ratios from the cancer cell line/normal human groups were analyzed to look for common differentially expressed genes in the different cell lines examined. Only the genes with a ratio of more than 5-fold increases (up-regulated in tumor versus normal group of the respective cancer) were considered for further analyses.

### 6. Selection of Tumor Biomarkers

In addition to the above analysis and the fold difference of up-regulated genes for each cancer, each of the up-regulated gene was selected only if the fold ratio was higher than 5 in the at least two tumor cell lines (e.g., for breast cancer, the two cell lines were MCF7 and MDA; for ovarian cancer, the three cell lines were SKOV3, 2008, and OVCAR-3; for colorectal cancer the two cell lines were T84 and HCT116; for lung cancer, the two cell lines were H460 and A549; for prostate cancer only one cell line was used, PC3 cells).

Five biomarkers were selected to fit the selection criteria based on up-regulated genes in all the cancerous cell lines tested on the 44K Agilent oligoarray. These five biomarkers, up-regulated by at least 5-fold, are referred to as “PAN” Cancer Biomarkers: TTK, SLC7A5, TRIM59, UHRF1 and KIF20A.

Table 2 shows the levels of KIF20A gene expression in cancer cell lines (e.g., for breast cancer, the two cell lines were MCF7 and MDA; for ovarian cancer, the three cell lines were SKOV3, 2008, and OVCAR-3; for colorectal cancer the two cell lines were T84 and HCT116; for lung cancer, the two cell lines were H460 and A549; for prostate cancer PC3 cells were used.

7. Validation of the PAN Biomarkers mRNA Expression

Validation of the level of mRNA expression of the PAN biomarkers in the different cancers was done by relative quantification using quantitative Real-Time PCR. In brief, the delta-delta Ct method was used where the expression levels of the PAN biomarkers are quantified relative to the lung H23 adenocarcinoma cells, normalized to an exogenous reference gene (from Arabidopsis thaliana) and adjusted by taking into account the efficiencies of the PAN biomarkers and reference gene primers. Different aspects of the Real-Time PCR assay were optimized before the PAN Biomarkers mRNA levels in the different cancerous tissues were measured.

### 8. Quantitative Real-Time PCR Assay

The methodology used for the quantitative Real-Time PCR assay and that used for all the set-up and validation of the assay is as follows: 500 ng of total RNA was mixed with 250 μg of pdN₆ random primers (GE Healthcare, Piscataway, N.J.), and 10 pg of Arabidopsis thaliana RNA, followed by 10 min incubation at 65° C. Samples were then cooled on ice for 2 min, and mixed with the cDNA synthesis solution to final concentrations of 50 mM Tris-HCl pH 8.3, 75 mM KCL, 3 mM MgCL₂, 10 mM DTT, 1 nM dNTP (Roche Diagnostics, Canada), and 200 units of Superscript III RT enzyme (Invitrogen, USA). The samples were then incubated at 25° C. for 5 min, and 1.5 hr at 50° C. As a RT reaction control, 10 pg of RNA from Arabidopsis thaliana was added to each sample. When amplified by real-time PCR, the specific Arabidopsis thaliana gene is expressed at a known levels (Ct between 19 and 20), and therefore ensures that all RT reactions worked the same. That prevents the usage of a housekeeping gene to control for the amount of cDNA. For each sample, a No RT reaction was also performed, omitting the Superscript III enzyme. This ensures that no genomic DNA was present in the total RNA preparations. The optimal annealing temperature was 60° C. for KIF20A. The Applied Biosystem taqman probes system (Foster City, USA) with the Light Cycler 480 (Roche Diagnostics, Canada) was used for this validation study. The reactions were prepared as followed: 10 μl Master Mix (final concentration of 1×), 1 μl taqman probe (final concentration of 1×), 4 μl of Rnase/Dnase-free water (Ambion, Canada), and 5 μl of cDNA or 5 μl of water (for No Template Control reactions) were added to each well for a final volume of 20 μl. As a reference sample, a calibrator of total RNA was prepared from the H23NSCLC adenocarcinoma cell line. This calibrator was used in each experiment, and the ratios to calibrator were calculated. This allowed for direct comparison between different experiments. In each test, duplicate wells were used for different controls to ensure that all reactions were reliable. “No Template” controls and “No RT” controls were included, an Arabidopsis thaliana gene was amplified, (as a normalization gene) and a calibrator sample was used to examine for consistency and accuracy.

The delta-delta Ct calculation method was used to analyze the real-time PCR data. Using this method, the cDNA synthesis and mRNA level are normalized with a calibrator (H23 total RNA). Briefly, the ddCt calculation compares the target gene Ct of each sample to the Ct of the calibrator for the same gene. This gives a ratio of expression relative to the calibrator (“referred to here as “the Normalized qPCR ratio”) and allows for comparison of the samples between experiments. The calibrator also accounts for the quality of the real-time experiment as it is always expressed at the same level in all genes tested. The mathematical equation for the relative quantification corrected for the efficiencies of the PAN biomarkers is as follows.

$$R = \frac{{( {E}_{target} )}^{Δ CPtarget ( control - sample )}}{{( {E}_{ref} )}^{Δ CPref ( control - sample )}}$$

### Example 2

### Ouantitative Real-Time PCR Assays Setup

### 1. Preparation of the Total RNA Calibrator

To determine the exact levels of expression of each PAN biomarker by quantitative Real-Time PCR, a calibrator cell line was used to which biomarker expression levels for each gene in patient tissues is compared to under identical reaction conditions. The calibrator was used in each experiment and allowed the comparison of different experiments. A representative range of Ct values were sought that could allow the proper quantification of each biomarker expression levels in patient samples. Preliminary experiments were done with two lung cell lines, the H23 adenocarcinoma (NSCLC) and the HFL-1 embryonic lung fibroblast cell lines. The two lung cell lines were cultured from frozen stocks in the absence of antibiotics in F-12K Nutrient mix (HFL-1 cells) or modified RPMI media (H23 cells). RNA was extracted from cells collected at various passages using the commercial RNeasy Mini Kit (Qiagen, USA). Gene expression levels for each of the biomarkers were tested in a two-step qRT-PCR, Reverse transcription and qPCR reaction was conducted as described previously. Under the conditions tested, the two tumor cell lines showed a good range for gene expression levels. For the purpose of this work, the H23 adenocarcinoma cells were selected.

### 2. Verification of the Probes Specificity and Primers Specificities

Real-Time PCR reaction products saved from the calibrator testing above were resolved on 2% agarose gels to verify the primers/probe specificity in both H23 and HFL-1 cell lines. A 60° C. PCR annealing temperature was optimal for SLC7A5, UHRF1, and KIF20A, however multiple bands were seen with TTK and TRIM59 primers. The latter multiple bands were resolved by increasing the annealing temperature to 62° C. and 64° C. which increase primer binding stringency for TTK and TRIM59 primers.

### 3. Assay Optimization

Following probe optimization, a small batch of H23 total RNA calibrator was prepared to verify the conditions of RNA extraction and DNAse treatment (i.e., the complete removal of genomic DNA (gDNA) from the RNA preparation). Three out of six reverse transcription reaction lacking the RT enzyme (no RT controls) gave a fluorescence signals. Moreover, DNA gel electrophoresis of the qRT-PCR products showed high molecular weight amplicons in the not RT controls, indicative of the persistence of gDNA. A 45 min DNAse digestion was done and DNA gel electrophoresis showed the disappearance of the high molecular weight amplicons. Using these latter optimized conditions, a large amount of total RNA was extracted from the H23 cells for cDNA calibrator preparation.

Using H23 cDNA preparation, standard curves of multiple replicates for each data point were set-up across a 10-fold serial dilution of the H23 cDNA (1:1 to 1:10,000). Using these standard curves, the amplification efficiencies and optimal qPCR annealing temperatures for each of the five PAN biomarker primers, including those for the Arabidopsis thaliana reference gene, were optimized. The standard curves were used to calculate the normalized ratio of each patient and to generate primer efficiencies, which correct the equation for relative quantification. Roche LightCycler 480 software was used to generate plots of Ct versus log of the dilution, and the slope of the line was used to calculate primer efficiencies using the equation E=10-1/slope-1. Five taqman probe/primers sets had acceptable efficiencies of between 1.78 and 2.2, and errors of less than 0.2.

### 4. Optimization of Patient Total RNA Required for Real-Time PCR:

To determine the optimal quantity of patient RNA to be tested (i.e., the amount that will give Ct values that lie within the standard curves), RNA samples from one NSCLC patient and one normal lung individual were quantified by Nanoprop to obtain 100 ng, 250 ng, and 500 ng of total RNA. Separate reverse transcription reactions were set-up as described above for each of these three quantities of RNA for both patient samples, and qPCR was performed on the six samples using the five optimized primer/probes combinations. Expression levels from the six samples were inspected to determine which of the three starting total RNA amounts (in nanograms) are within range of the Cts covered by each PAN biomarker standard curve. 500 ng is the optimal quantity of patient RNA for reverse-transcription qPCR in order to obtain Ct values that could be accurately quantified by standard curves without having to extrapolate.

### Example 3

### Validation of the PAN Biomarkers in Clinical Samples

Five different groups of patients were studied. The lung cancer group consisted of non-small cell lung cancer (NSCLC) patients with a variety of subtypes (mainly adenocarcinomas and squamous cell carcinomas. Patients within the lung cancer group had an average age of 62.5 years and were mostly male. Early disease stages were well represented (1-II) (with only one stage III patient) in this group samples. The Breast Cancer Group was of an average age of 53.1 years with a majority of Caucasian women. Stages I and II breast cancer are equally represented in this group, as well as the women menopausal status. For the breast cancer patients, the majority of the cases were infiltrating ductal carcinoma. The Ovarian Cancer Group of patients was of an average age of 61.5 and patients diagnosed with serous adenocarcinomas stage III, mostly menopausal. The Colorectal Cancer Group, patients were only males with an average age of 69.7 years. Cases were distributed equally between stages I to III and were classified as adenocarcinoma of the colon. The Prostate Cancer Group, patients were of an average age of 62 years with stage II prostate cancer. The majority of patients were diagnosed with adenocarcinoma of the prostate.

The normal patients for each cancer were coming from different individuals (lung, breast and ovary) except for colon and prostate cases. For the latter two cancers, the normal samples were normal matched samples from the same patients.

For breast, ovarian and lung patients, total RNA samples were obtained from several tissue diposatories [Asterand Inc. (Detroit, USA), Clinomics Biosciences Inc (Watervliet, USA) and Biochain Institute Inc. (Hayward, USA). Total RNA was extracted from snap frozen tissues samples using Trizol Reagent kit (Gibco-BRL, USA) extraction procedure. Total RNA was treated with RNA-free DNAse I and purified with the RNEasy kit (Qiagen, USA). RNA samples were visualized and analyzed on an Agilent 2100 BioAnalyzer (Agilent, USA) for purity and integrity.

For the colorectal and prostate cancers, patients samples were obtained from Indivumed Inc (Hamburg, Germany) as 10 μm formalin-fixed paraffin embedded (FFPE) sections. Total RNA was extracted from FFPE section using the High pure RNA paraffin kit (Roche) with some modifications. Briefly, the paraffin sections were deparaffinized by incubation in Citrosolv (Fisher) for 10 min and washed 2 times with 99% ethanol for 10 min. After the final wash, the paraffin sections were scratch and the material was air-dried at 55° C. for 10 min. Each sample was incubated with 100 μl Tissue Lysis Buffer, 16 μL 10% SDS and 40 μL proteinase K, homogenized and incubated overnight at 55° C. After proteinase K digestion, RNA was isolated by the addition of 325 μl Binding Buffer and 325 μl ETOH 99% and gently mixed. The lysate was added to the column and centrifuged at 8,000 rpm for 30 sec, at RT. The sample was dried completely by centrifugation at 12,000 rpm for 30 sec, and washed with 500 μl Wash Buffer I, followed by two washed with Wash buffer II. After each wash, the sample was centrifuged at 8000 g for 20 sec and the flow through was discarded. A last centrifugation was done at 12,000 rpm for 2 min to ensure that the entire buffer was removed. RNA was eluted with 90 μl of elution buffer, by incubation for one min at RT, and a centrifugation at 8000 g for 1 min. To remove genomic DNA, all samples were incubated with 2 μl of DNase 5 U/μl (Roche) at 37° C. for 1 hr. After the DNase treatment, the sample were homogenized and incubated in digestion buffer with proteinase K (20 μl Tissue Lysis Buffer, 10% SDS 40 μl, Proteinase K) at 55° C. for 1 hr. RNA was isolated, washed and collected by centrifugation after incubation at RT for 1 min with 50 μl of elution buffer. Lastly, the amount of RNA in the samples was measured using the Nanodrop® ND-1000 spectrophotometer. The purity of the RNA extracted from each FFPE tissue samples was evaluated by the 260/280 ratio obtained during the RNA quantification (Nanodrop® ND-100 spectrophotometer).

### Example 4

### Receiver Operating Characteristic (ROC) Curves

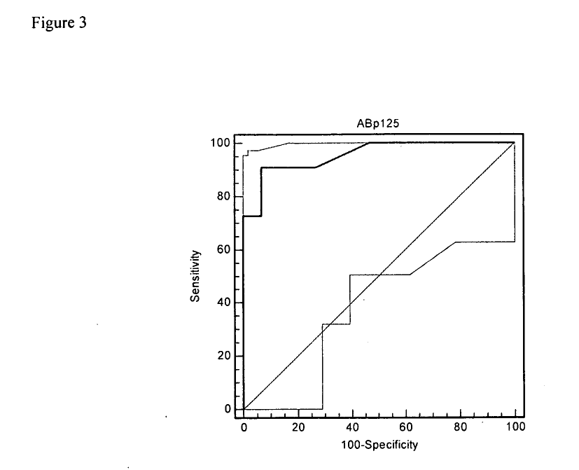

Receiver operating curves were done with the MedCal software using the normalized qPCR ratios obtained during the qRT-PCR analyses of each PAN biomarkers on the panel of cancerous patients tested. Each cancer was analyzed separately. ROC curves were generated for each biomarker and area under the curve (AUC), sensitivity and specificity were obtained. Further analyses were done using the cut-off value obtained under the high accuracy setting and using the cut-off value calculated by the software when the specificity of the assay is set to 100% (no false positive result). Combinations of PAN biomarkers were assessed using a scoring system based on the cut-off values (high accuracy and 100% specificity). In summary, for each patient, a score of 1 was given when the ratio obtained for the biomarker was superior to the cut-off value of that biomarker. Then, for each patient, a sum of the score obtained for each target was compiled and used for the ROC curve analysis. The results are shown in FIG. 3 (lung), 6 (breast), 8 (ovarian), and 10 (colorectal).

### Example 5

### Real-Time Quantitive PCR for the Dectection KIF20A in Samples Obtained from Normal Lung Subjects and Lung Cancer Patients

1. Total RNA Isolation and cDNA Labeling

Patient tissues samples were obtained from Asterand, Inc. (Detroit, Mich.), and Biochain Institute, Inc. (Hayward, Calif.). Each patient included in the study was screened against the same normal total RNA group in order to compare them together. The tumor group was composed of 11 cases. The lung normal group was composed of 15 cases.

### 2. Results

Increased levels of KIF20A mRNA were detected in tumor fluid and cell samples obtained patients suffering from non-small lung cancer compared to the levels in fluid and cell samples obtained from normal lung subjects (FIG. 1). Tumor samples from patients suffering from lung cancer averaged about 13 times higher levels of KIF20A mRNA expression than found in normal subjects (FIG. 2 and Table 1). These results establish that KIF20A is a marker of neoplastic disease in lung.

Similarly, the differential expression of all TTK, SLC7A5, TRIM59 and KIF20A mRNAs was measured in the same NSCLC patients using quantitative Real-Time PCR technique. In comparison to the other cancers tested, the fold increase measured in the lung cancer are high for all five PAN biomarkers and may reflect the results seen with the whole human genome studies in cancerous cell lines.

To determine the predictive values of measuring the differential expression of KIF20A, alone and in combination with TTK, SLC7A5, TRIM59 and/or UHRF1 for lung cancer, the expression levels of these PAN biomarkers RNAs were analyzed using the method of Receiver Operating Characteristic (ROC) curves. ROC curves analyses were done for each PAN biomarker separately and in combination. For NSCLC samples, a good area under the curve (AUC) was obtained for KIF20A (FIG. 3) and for each of the other four PAN biomarkers. With the high accuracy cut-off value, sensitivity and specificity was obtained for all the PAN biomarkers. However, when the cut-off values selected are the ones that give 100% specificity, the sensitivity decreased to 72.7 to 81.8%. Perfect AUC (100%) is obtained when all the PAN biomarkers are combined at high accuracy (at least two biomarkers is over their cut-off values) but decrease to 96% when there is 100% specificity (sensitivity of 90.9%). In that case, the score need to be of at least one, meaning that only one biomarker has a normalized qPCR ratio over its cut-off value, as shown in Table 4.

TABLE 4

| | | High Accuracy | | | | 100% Specificity | | |
|---|---|---|---|---|---|---|---|---|
| | Auc | Sensitivity | Specificity | Cut-off | Auc | Sensitivity | Specificity | Cut-off |
| KIF20A | 0.96 | 90.9 | 93.3 | >0.05 | | 72.7 | 100 | >0.20 |
| SLC7A5 | 0.99 | 100 | 86.7 | >0.02 | | 81.8 | 100 | >0.03 |
| TRIM59 | 0.90 | 91 | 93.3 | >0.17 | | 81.8 | 100 | >0.19 |
| TTK | 0.95 | 81.8 | 100 | >0.06 | | 81.8 | 100 | >0.06 |
| UHRF1 | 0.98 | 100 | 93.3 | >0.01 | | 72.7 | 100 | >0.06 |
| KIF20A + SLC7A5 | 0.99 | 90.9 | 100.0 | >score 1 | 0.91 | 81.8 | 100 | >score 0 |
| KIF20A + TRIM59 | 0.99 | 100.0 | 86.7 | >score 0 | 0.95 | 90.9 | 100 | >score 0 |
| KIF20A + TTK | 0.91 | 90.9 | 100 | >score 0 | 0.91 | 81.8 | 100 | >score 0 |
| KIF20A + UHRF1 | 1 | 100.0 | 100 | >score 0 | 0.91 | 81.8 | 100 | >score 0 |
| SLC7A5 + TRIM59 | 0.96 | 90.9 | 80.0 | >score 1 | 0.95 | 90.9 | 100 | >score 0 |
| SLC7A5 + TTK | 1 | 100 | 100 | >score 0 | 0.91 | 82 | 100 | >score 0 |
| SLC7A5 + UHRF1 | 1 | 100.0 | 93.3 | >score 1 | 0.91 | 81.8 | 100 | >score 0 |
| TRIM59 + TTK | 0.99 | 100 | 86.7 | >score 0 | 0.95 | 90.9 | 100 | >score 0 |
| TRIM59 + UHRF1 | 1 | 100 | 93.3 | >score 0 | 0.95 | 90.9 | 100 | >score 0 |
| TTK + UHRF1 | 1 | 100.0 | 100 | >score 0 | 0.91 | 81.8 | 100 | >score 0 |
| PAN (5) | 1 | 100 | 100 | >score 1 | 0.96 | 90.9 | 100 | >score 0 |
| Potentially | 0.96 | 90.9 | 80.0 | >score 1 | 0.95 | 90.9 | 100 | >score 0 |
| Secreted (2) | | | | | | | | |

### Example 6

### Western Blot Analysis of Samples Isolated from Lung Cancer Patients and Normal Lung Subjects

### 1. Patient Samples and Normal Samples

Patient lung tissues and pleural fluid samples are obtained from Asterand, Inc. (Detroit, Mich.), and Biochain Institute, Inc. (Hayward, Calif.). Each patient included in the study is screened against the same normal total RNA group in order to compare them together.

### 2. Western Blot Analysis of KIF20A in Lung Cancer and Lung Normal Samples

Fluid samples are prepared by in one of two ways: a) mixing total unfractionated pleural fluid with lysis buffer as described below; or b) the pleural fluid is first fractionated by centrifugation where both the pellet and supernatant material are mixed with lysis buffer. Protein lysates from a) and b) are then quantified and equal amounts of protein are resolved on SDS-PAGE and Western blotting.

For lung cell samples, human tissues are homogenized using a Polytron PT10-35 (Brinkmann, Mississauga, Canada) for 30 sec at speed setting of 4 in the presence of 300 μl of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada).

40 μg of proteins from human lung tissue samples and fluid samples isolated from cancer patients and normal lung subjects are used in SDS-PAGE gels. Samples are mixed with Laemmli buffer, heated for 5 min at 95° C., and then resolved by 12% SDS-PAGE. Proteins are then electro-transferred onto Hybond-ECL nitrocellulose membranes (Amersham Biosciences, Baie d'Urfe, Canada) for 90 min at 100 volts at RT. Membranes are blocked for 1 hr at RT in blocking solution (PBS containing 5% fat-free dry milk). Membranes are washed with PBS and are incubated with the primary anti-KIF20A antibodies at the appropriate dilutions in blocking solution containing 0.02% sodium azide for 2 hr at RT. PBS washing is performed, and the membranes are subsequently incubated for 1 hr at RT with secondary anti-mouse, anti-rabbit or anti-goat antibodies labeled with horseradish peroxydase (Bio-Rad, Mississauga, Canada) diluted 1/3000 in PBS. Chemiluminescence detection is performed using the SuperSignal West Pico Chemiluminescent Substrate (Pierce, Rockford, Ill., USA) following the manufacturer's recommendations.

### 3. Results

KIF20A expression is significantly increased in cell and fluid samples obtained from lung tumor patients as compared to expression in cell and fluid samples isolated from normal subjects. All normal subjects show nearly undetectable levels of KIF20A protein expression, while samples obtained from lung cancer patients show detectable levels of KIF20A.

### Example 7

### Real-Time Quantitive PCR for the Detection of KIF20A in Samples Obtained from Normal Breast Subjects and Breast Cancer Patients

1. Total RNA Isolation and cDNA Labeling

Patient tissues samples were obtained from Asterand, Inc. (Detroit, Mich.), Clinomics Biosciences, Inc (Watervliet, N.Y.) and Biochain Institute, Inc. (Hayward, Calif.). Each patient included in the study was screened against the same normal total RNA group in order to compare them together. The tumor group was composed of 17 cases. The breast normal group was composed of 10 cases.

### 2. Results

Increased levels of KIF20A mRNA were detected in tumor fluid and cell samples obtained patients suffering from breast cancer compared to the levels in fluid and cell samples obtained from normal breast subjects (FIGS. 4-5). Tumor samples from patients suffering from breast cancer averaged about 13-fold higher levels of KIF20A mRNA expression than found in normal subjects (Table 1). These results establish that KIF20A is a marker of neoplastic disease in breast.

Similarly, the differential expression of all four biomarkers (e.g., TTK, SLC7A5, TRIM59 and UHRF1) mRNAs was measured in the same breast patients using quantitative Real-Time PCR technique. The results show significant differences in RNA expression for each of the PAN biomarkers between the breast samples and normal breast samples from patients.

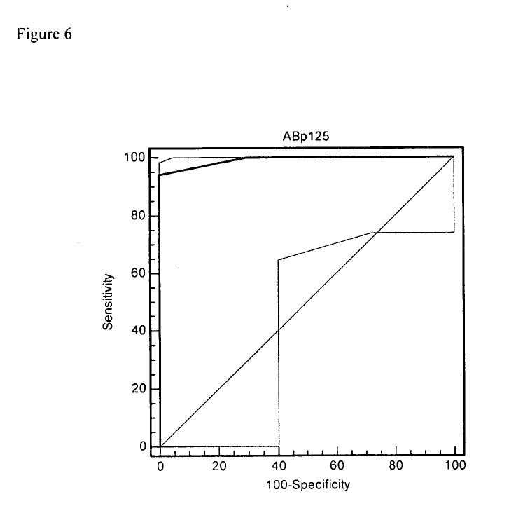

To determine the predictive values of measuring the differential expression of KIF20A, alone and in combination with TTK, SLC7A5, TRIM59 and/or UHRF1 for breast cancer, the expression levels of these PAN biomarkers RNAs were analyzed using ROC curves. ROC curves analyses were done for each PAN biomarker separately (FIG. 6 for KIF20A) and in combination. The results in Table 5 summarize the performances of KIF20A and the other biomarkers in breast cancer samples.

TABLE 5

| | | High Accuracy and 100% Specificity | | |
|---|---|---|---|---|
| Target | Auc | Sensitivity | Specificity | Cut-off |
| KIF20A | 0.991 | 94.12 | 100 | >0.02 |
| SLC7A5 | 0.982 | 88.24 | 100 | >0.02 |
| TRIM59 | 1 | 100 | 100 | >0.13 |
| TTK | 0.994 | 94.12 | 100 | >0.03 |
| UHRF1 | 1 | 100 | 100 | >0.01 |
| KIF20A + SLC7A5 | 1 | 100 | 100 | >score 0 |
| KIF20A + TRIM59 | 1 | 100 | 100 | >score 0 |
| KIF20A + TTK | 0.97 | 94.1 | 100 | >score 0 |
| KIF20A + UHRF1 | 1 | 100 | 100 | >score 0 |
| SLC7A5 + TRIM59 | 1 | 100 | 100 | >score 0 |
| SLC7A5 + TTK | 1 | 100 | 100 | >score 0 |
| SLC7A5 + UHRF1 | 1 | 100 | 100 | >score 0 |
| TRIM59 + TTK | 1 | 100 | 100 | >score 0 |
| TRIM59 + UHRF1 | 1 | 100 | 100 | >score 0 |
| TTK + UHRF1 | 1 | 100 | 100 | >score 0 |
| PAN (5) | 1 | 100 | 100 | >score 0 |
| Potentially secreted (2) | 1 | 100 | 100 | >score 0 |

### Example 8

### Western Blot Analysis of Samples Isolated From Breast Cancer Patients and Normal Breast Subjects

### 1. Patient Samples and Normal Samples

Patient breast tissues and pleural fluid samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA group in order to compare them together.

### 2. Western Blot Analysis of KIF20A in Breast Cancer and Breast Normal Samples

Fluid samples are prepared by in one of two ways: a) mixing total unfractionated pleural fluid with lysis buffer as described below; or b) the pleural fluid is first fractionated by centrifugation where both the pellet and supernatant material are mixed with lysis buffer. Protein lysates from a) and b) are then quantified and equal amounts of protein are resolved on SDS-PAGE and Western blotting.

For breast cell samples, human tissues are homogenized using a Polytron PT10-35 (Brinkmann, Mississauga, Canada) for 30 sec at speed setting of 4 in the presence of 300 μl of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada).

40 μg of proteins from human breast tissue samples and fluid samples isolated from cancer patients and normal breast subjects are used in SDS-PAGE gels. Samples are mixed with Laemmli buffer, heated for 5 min at 95° C., and then resolved by 12% SDS-PAGE. Proteins are then electro-transferred onto Hybond-ECL nitrocellulose membranes (Amersham Biosciences, Baie d'Urfé, Canada) for 90 min at 100 volts at RT. Membranes are blocked for 1 hr at RT in blocking solution (PBS containing 5% fat-free dry milk). Membranes are washed with PBS and are incubated with the primary anti-KIF20A antibodies at the appropriate dilutions in blocking solution containing 0.02% sodium azide for 2 hr at RT. PBS washing is performed, and the membranes are subsequently incubated for 1 hr at RT with secondary anti-mouse, anti-rabbit or anti-goat antibodies labeled with horseradish peroxydase (Bio-Rad, Mississauga, Canada) diluted 1/3000 in PBS. Chemiluminescence detection is performed using the SuperSignal West Pico Chemiluminescent Substrate (Pierce, Rockford, Ill., USA) following the manufacturer's recommendations.

### 3. Results

KIF20A expression is significantly increased in cell and fluid samples obtained from breast tumor patients as compared to expression in cell and fluid samples isolated from normal subjects. All normal subjects show nearly undetectable levels of KIF20A protein expression, while samples obtained from breast cancer patients show detectable levels of KIF20A.

### Example 9

### ELISA Analysis of KIF20A in Breast Cancer and Breast Normal Tissues

### 1. Isolation and Preparation of Patient and Normal Tissues

Patient tissue samples were obtained and prepared as described in Example 7.

### 2. ELISA Analysis

To quantify the amount of each target of interest and to confirm the results obtained by Western blot, an ELISA technique was performed on ovarian samples for KIF20A. Prior to screening all samples, an optimization of the conditions was performed using normal and tumor samples to determined the linearity of the assay (dose-dependant curve, time of development of the assay). Once conditions were optimized (Results to come), 96-well plates ((Maxisorp plates, NUNC, (Rochester, N.Y., USA)) were coated with the capture antibody. Samples were then incubated overnight at 4° C. Wells were washed 3 times with PBS and then blocked with bovine serum albumin (BSA)/PBS or BSA alone for 1 hr at RT. Detection antibodies (40 ng/well) were added to the wells and incubated for 2 hr at RT. Plates were washed 3 times with PBS and the secondary anti-mouse, anti-rabbit or anti-goat antibodies labeled with horseradish peroxidase (Bio-Rad, Mississauga, Canada), diluted 1:3000 in 3% BSA/PBS, was incubated for 1 hr at RT. Wells were washed 3 times with PBS and developed with 2,2′-azino-bis(3-ethylbenzthiazoline-6-sulfonic acid) (ABTS) as the substrate (Sigma Corp., St. Louis, Mo.).

The intensity of the signal was assessed by reading the plates at a 405 nm wavelength using a microplate reader. For each of the target, a standard curve was established with a recombinant or purified protein at the same time to quantify the target in each sample. Results were expressed as concentrations of a target in 1 μg of total protein extract. All samples were quantified in the same assay. Differences among normal and tumor groups were analyzed using Student's two-tailed t test with significance level defined as P<0.05.

### 3. Results

ELISA results show the levels of KIF20A protein expression in normal and breast tissue samples. Results are shown as ng/μg of protein marker in each normal subject versus ng/μg of protein marker in each breast cancer patient. These results confirm the results obtained in the Western blot protein analysis.

### Example 10

### Real-Time Quantitative PCR for the Detection of KIF20A in Samples Obtained from Normal Ovarian Subjects and Ovarian Cancer Patients

1. Total RNA Isolation and cDNA Labeling

Patient tissues samples were obtained from Asterand, Inc. (Detroit, Mich.), Clinomics Biosciences, Inc (Watervliet, N.Y.) and Biochain Institute, Inc. (Hayward, Calif.). Each patient included in the study was screened against the same normal total RNA group in order to compare them together. The tumor group was composed of 17 cases. The ovarian normal group was composed of 10 cases.

### 2. Results

Increased levels of KIF20A mRNA were detected in tumor fluid and cell samples obtained patients suffering from ovarian cancer compared to the levels in fluid and cell samples obtained from normal ovarian subjects (FIG. 7). Tumor samples from patients suffering from ovarian cancer averaged about 3-fold higher levels of KIF20A mRNA expression than found in normal subjects (Table 1). These results establish that KIF20A is a marker of neoplastic disease in ovarian.

Similarly, the differential expression of all four biomarkers (e.g., TTK, SLC7A5, TRIM59 and KIF20A) mRNAs was measured in the same ovarian patients using quantitative Real-Time PCR technique. There is a significant differences in RNA expression for each of the PAN biomarkers between the ovarian test samples and normal ovarian samples from patients.

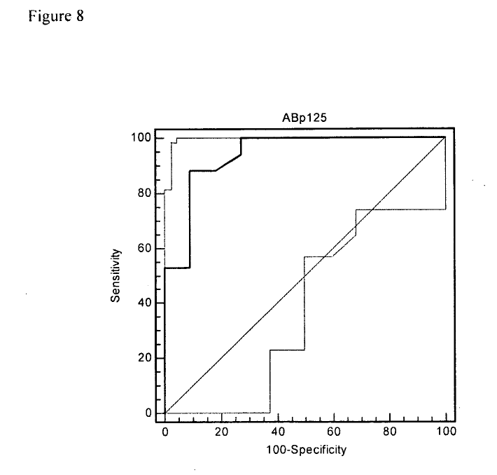

To determine the predictive values of measuring the differential expression of KIF20A, alone and in combination with TTK, SLC7A5, TRIM59, and/or UHRF1 for breast cancer, the expression levels of these PAN biomarkers RNAs were analyzed using ROC curves. ROC curves analyses were done for each PAN biomarker separately (KIF20A: FIG. 8) and in combination (Table 6).

TABLE 6

| | | High Accuracy | | | | 100% Specificity | | |
|---|---|---|---|---|---|---|---|---|
| Target | Auc | Sensitivity | Specificity | Cut-off | Auc | Sensitivity | Specificity | Cut-off |
| KIF20A | 0.94 | 88.2 | 90.9 | >0.21 | | 52.9 | 100 | >0..46 |
| SLC7A5 | 0.84 | 76.5 | 81.8 | >0..03 | | 29.4 | 100 | >0.13 |
| TRIM59 | 0.98 | 94.1 | 100.0 | >0.7 | | 94.1 | 100 | >0.7 |
| TTK | 0.995 | 94.1 | 100 | >0.1 | | 94.1 | 100 | >0.1 |
| UHRF1 | 0.85 | 100 | 72.7 | >0.009 | | 29.4 | 100 | >0.12 |
| KIF20A + SLC7A5 | 0.90 | 94.1 | 81.8 | >score 0 | 0.77 | 52.9 | 100 | >score 0 |
| KIF20A + TRIM59 | 0.97 | 88.2 | 100.0 | >score 1 | 0.97 | 94.1 | 100 | >score 0 |
| KIF20A + TTK | 0.97 | 88.2 | 100 | >score 1 | 0.97 | 94.1 | 100 | >score 0 |
| ABp125 + 129 | 0.84 | 88.2 | 82 | >score 0 | 0.79 | 58.8 | 100 | >score 0 |
| SLC7A5 + TRIM59 | 0.97 | 100.0 | 81.8 | >score 0 | 0.97 | 94.1 | 100 | >score 0 |
| SLC7A5 + TTK | 0.97 | 100 | 82 | >score 0 | 0.97 | 94 | 100 | >score 0 |
| SLC7A5 + UHRF1 | 0.79 | 88.2 | 72.7 | >score 0 | 0.91 | 81.8 | 100 | >score 0 |
| TRIM59 + TTK | 0.91 | 94.1 | 81.8 | >score 0 | 0.77 | 52.9 | 100 | >score 0 |
| TRIM59 + UHRF1 | 0.91 | 94 | 81.8 | >score 0 | 0.97 | 94.1 | 100 | >score 0 |
| TTK + UHRF1 | 0.91 | 94.1 | 82 | >score 0 | 0.97 | 94.1 | 100 | >score 0 |
| PAN (5) | 0.98 | 94 | 91 | >score 1 | 0.97 | 94.1 | 100 | >score 0 |
| Potentially Secreted (2) | 0.97 | 100 | 82 | >score 0 | 0.97 | 94.1 | 100 | >score 0 |

### Example 11

### Western Blot Analysis of Samples Isolated from Ovarian Cancer Patients and Normal Ovarian Subjects

### 1. Patient Samples and Normal Samples

Patient tissue samples were obtained from Asterand, Inc. (Detroit, Mich.), Clinomics Biosciences, Inc (Watervliet, N.Y.) and Biochain Institute, Inc. (Hayward, Calif.). The samples were isolated from normal ovaries and ovarian cancer tissues, and were frozen into blocks of tissue. Protein cell extracts were then prepared from each block. Each patient included in the study was screened against the same normal total RNA group in order to compare them together. The tumor group composed of 36 cases. The ovarian normal group was composed of 34 cases.

### 2. Western Blot Analysis of KIF20A in Ovarian Cancer and Normal Ovarian Samples

For ovarian cell samples, human tissues were homogenized using a Polytron PT10-35 (Brinkmann, Mississauga, Canada) for 30 sec at speed setting of 4 in the presence of 300 μl of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada). 40 μg of proteins from human ovarian cancer patients and normal ovarian subjects were used in SDS-PAGE gels. Samples were mixed with Laemmli buffer (250 mM Tris-HCl, pH 8.0, 25% (v/v) b-mercaptoethanol, 50% (v/v) glycerol, 10% (w/v) SDS, 0.005% (w/v) bromophenol blue), heated for 5 min at 95° C. and resolved in 12% SDS-polyacrylamide gels (SDS-PAGE). Proteins were then electro-transferred onto Hybond-ECL nitrocellulose membranes (Amersham Biosciences, Baie d'Urfé, Canada) for 90 min at 100 volts at RT. Membranes were blocked for 1 hr at RT in blocking solution (PBS containing 5% fat-free dry milk). Membranes were washed with PBS and incubated with the primary anti-KIF20A polyclonal antibodies or monoclonal antibodies at the appropriate dilutions in blocking solution containing 0.02% sodium azide for 2 hr at RT. Antibodies were produced in house. PBS washing was performed, and the membranes were subsequently incubated for 1 hr at RT with secondary anti-mouse, anti-rabbit or anti-goat antibodies labeled with horseradish peroxydase (Bio-Rad, Mississauga, Canada) diluted 1/3000 in PBS. Chemiluminescence detection was performed using the SuperSignal West Pico Chemiluminescent Substrate (Pierce, Rockford, Ill., USA) following the manufacturer's recommendations.

### 3. Results

KIF20A expression was significantly increased in tumor samples obtained from ovarian tumor patients as compared to expression in samples from normal subjects. All normal subjects showed nearly undetectable levels of KIF20A protein expression, while nearly 60% of samples obtained from ovarian cancer patients showed detectable levels of KIF20A.

### Example 12

### Real-Time Quantitative PCR for the Detection of KIF20A in Samples from Colon Cancer Patients and Normal Colon Subjects

### 1. Patient Samples and RNA Isolation

Total RNA extraction from tumor cell lines and patient samples is performed as described in Example 5.

### 2. Real-Time PCR

Real-time PCR and analysis of results are performed as shown in Example 2. ROC curves were prepared as described in Example 4.

### 3. Results

Increased levels of RNA expression are identified in colon tumor samples as compared to expression in normal colon samples. Normal colon samples show less RNA expression of KIF20A than do colon tumor samples. Level of the KIF20A biomarker mRNA was evaluated in a group of male colorectal cancer patients with stages ranging from 1 to III. KIF20A is up-regulated significantly in colorectal cancer patients compared to the normal samples (FIG. 13).

KIF20A shows a 7.1-fold increase in the level of up-regulation relative to normal colon samples.

As shown in Table 7, it can be seen that the majority of the PAN biomarkers have good AUC separately and in combination.

TABLE 7

| | | High Accuracy | | | | 100% Specificity | | |
|---|---|---|---|---|---|---|---|---|
| Target | Auc | Sensitivity | Specificity | Cut-off | Auc | Sensitivity | Specificity | Cut-off |
| KIF20A | 0.94 | 80.0 | 100.0 | >0.0036 | | 80.0 | 100 | >0.0036 |
| SLC7A5 | 1.00 | 100.0 | 100 | >0.0013 | | 100.0 | 100 | >0.0013 |
| TRIM59 | 0.90 | 80 | 90 | >0.0044 | | 60.0 | 100.0 | >0.0061 |
| TTK | 0.87 | 100.0 | 60.0 | >0.0022 | | 50.0 | 100.0 | >0.01 |
| UHRF1 | 0.96 | 90.0 | 100.0 | >0.0041 | | 90.00 | 100.00 | >0.0041 |
| KIF20A + SLC7A5 | 1 | 100.0 | 100.0 | >score 0 | 1.00 | 100 | 100 | >score 0 |
| KIF20A + TRIM59 | 0.90 | 80.0 | 100.0 | >score 0 | 0.90 | 80.0 | 100 | >score 0 |
| KIF20A + TTK | 0.95 | 80.0 | 100.0 | >score 1 | 0.90 | 80.0 | 100 | >score 0 |
| KIF20A + UHRF1 | 0.94 | 90.0 | 90.0 | >score 0 | 0.95 | 90 | 100 | >score 0 |
| SLC7A5 + TRIM59 | 1.00 | 100.0 | 100.0 | >score 0 | 1 | 100 | 100 | >score 0 |
| SLC7A5 + TTK | 1.00 | 100.0 | 100.0 | >score 1 | 1 | 100 | 100 | >score 0 |
| SLC7A5 + UHRF1 | 0.995 | 100.0 | 90.0 | >score 0 | 1 | 100 | 100 | >score 0 |
| TRIM59 + TTK | 0.90 | 60.0 | 100.0 | >score 1 | 0.80 | 60.0 | 100.0 | >score 0 |
| TRIM59 + UHRF1 | 0.93 | 90.0 | 90.0 | >score 0 | 0.95 | 90.0 | 100.0 | >score 0 |
| TTK + UHRF1 | 0.93 | 90.0 | 90.0 | >score 1 | 0.95 | 90.0 | 100.0 | >score 0 |
| PAN (5) | 0.995 | 100.00 | 90.0 | >score 1 | 1.00 | 100.0 | 100.0 | >score 0 |
| Potentially | 1.00 | 100.0 | 100.0 | >score 0 | 1.00 | 100.0 | 90.0 | >score 0 |
| Secreted (2) | | | | | | | | |

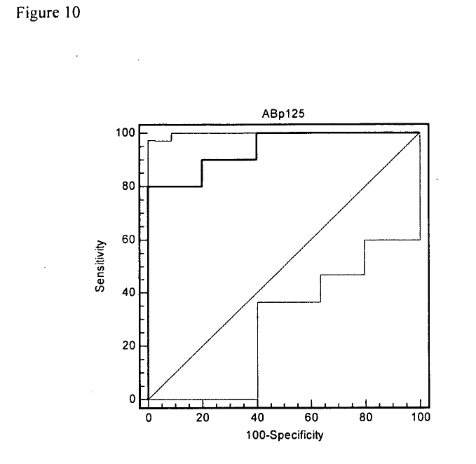

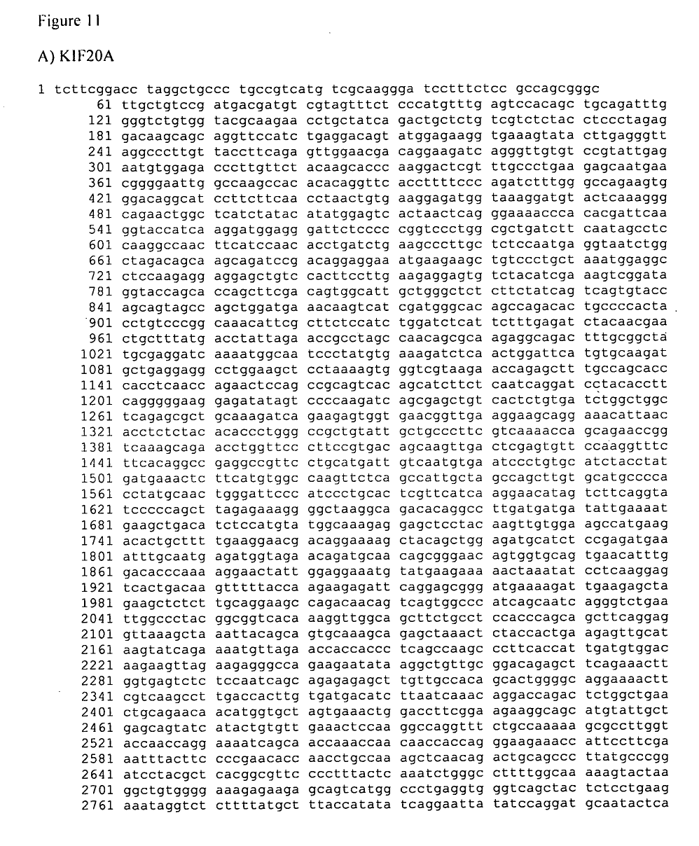

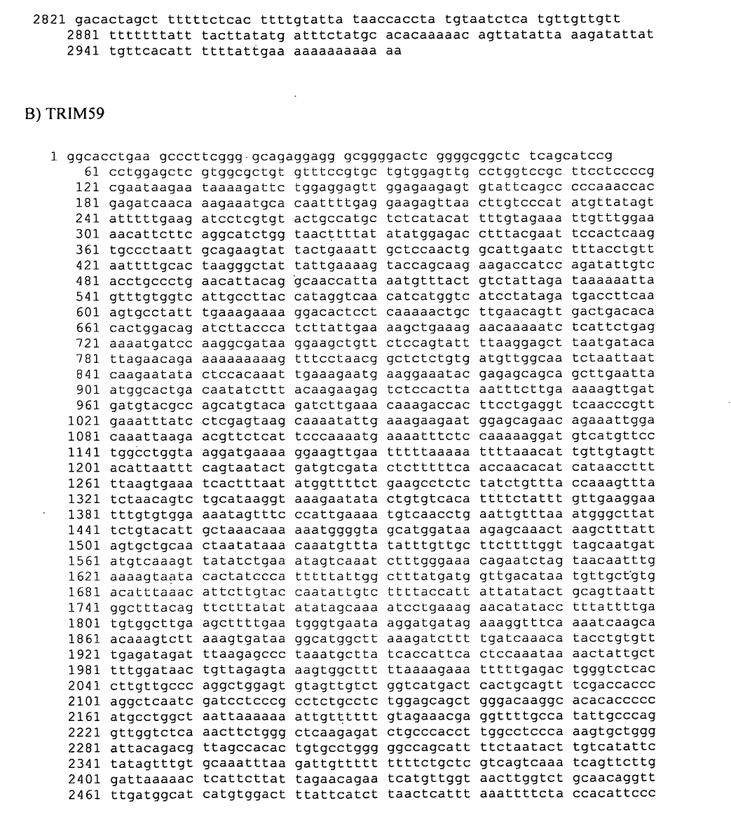

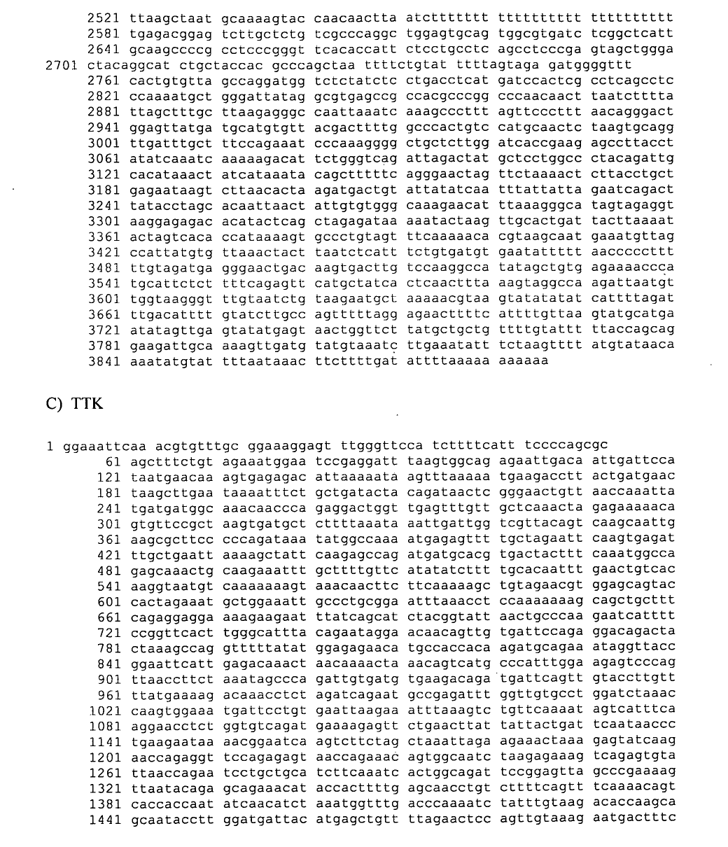

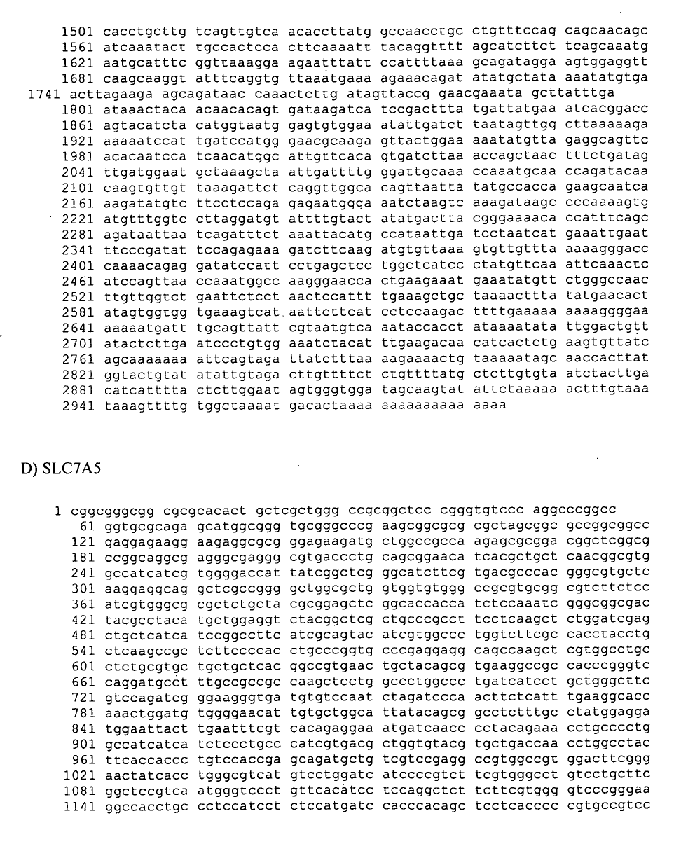

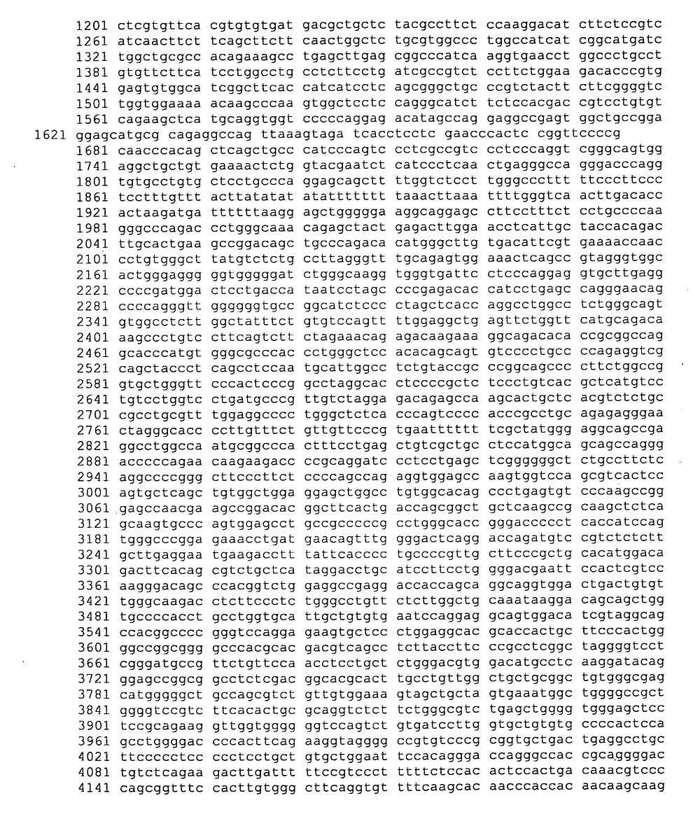

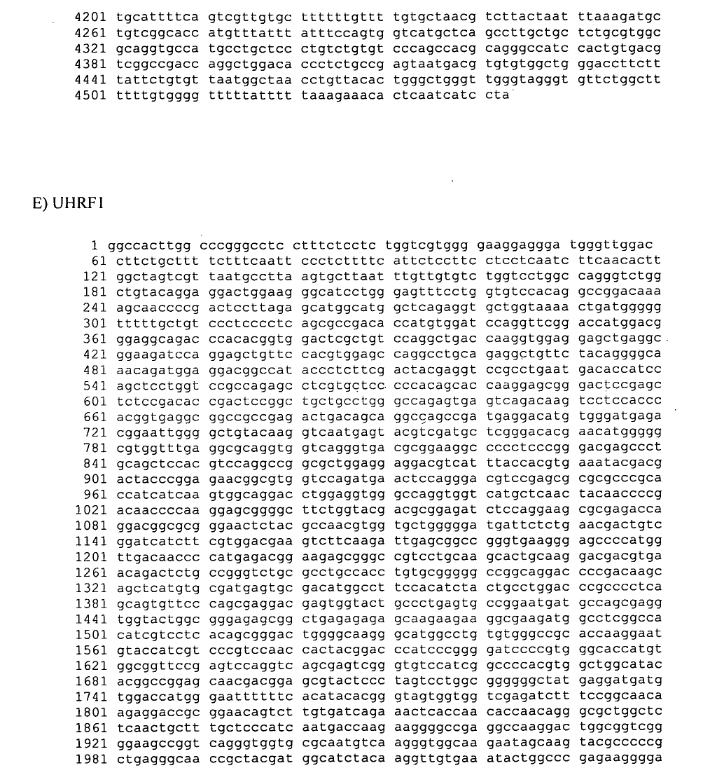

From Table 7, it can be seen that the majority of the PAN biomarkers have good AUC separately or in combination. The ROC curve for TTK is shown in FIG. 10. Some of the PAN biomarkers, two by two combinations, have a perfect AUC as seen for the potentially secreted targets. When the specificity is set to 100%, sensitivity drops from 50%-100% depending on if the PAN is alone, in two by two combinations or all together. In that case, sensitivity and specificity are 100% and only one biomarker need to be over the cut-off value (score>0).

### Example 13

### Western Blot Analysis of Samples Isolated from Colon Cancer Patients and Normal Colon Subjects

### 1. Patient Samples and Normal Samples

Patient tissue samples are obtained from Asterand, Inc. (Detroit, Mich.), Clinomics Biosciences, Inc (Watervliet, N.Y.) and Biochain Institute, Inc. (Hayward, Calif.). The samples are isolated from normal colon and colon cancer samples, and are frozen into blocks of tissue. Protein cell extracts are then prepared from each block. Each patient included in the study is screened against the same normal total RNA group in order to compare them together. The tumor group is composed of at least 20 cases. The colon normal group is composed of at least 20 cases.

### 2. Western Blot Analysis of KIF20A in Colon Cancer and Colon Normal Samples

Colon cell samples are isolated as described in Example 12. Western blot experiments are also performed as described in Example 11.

### 3. Results

KIF20A expression is significantly increased in tumor samples obtained from colon tumor patients as compared to normal samples isolated from normal subjects. All normal subjects show nearly undetectable levels of KIF20A protein expression, while samples obtained from colon cancer patients show detectable levels of KIF20A.

### Example 14

### ELISA Analysis of KIF20A in Colon Cancer and Colon Normal Tissues

### 1. Isolation and Preparation of Patient and Normal Tissues

Patient tissue samples are obtained and are prepared as described in Example 6.

### 2. ELISA Analysis

ELISA analysis is performed as described in Example 6.

### 3. Results

ELISA results show that samples from normal subjects expressed less KIF20A protein compared to colon cancer patient samples. These results confirm the results obtained in the Western blot expression.

### Example 15

### Western Blot Analysis of Samples Isolated from Leukemia Patients and Normal Subjects

### 1. Patient Samples and Normal Samples

Patient marrow tissues and blood are obtained from Asterand, Inc. (Detroit, Mich.), Clinomics Biosciences, Inc (Watervliet, N.Y.) and Biochain Institute, Inc. (Hayward, Calif.). Each patient sample included in the study is screened against the same normal total RNA group in order to compare them together.

### 2. Western Blot Analysis of KIF20A in Leukemia and Normal Samples

Blood samples are prepared by isolating blood from leukemia patients. The blood samples are fractioned initially to isolate remove red-blood cells. The samples containing all white blood cell are further fractionated by FACS sorting based on size defractions and/or using surface specific monoclonal antibodies. Purified cells are then lysed in lysis buffer as described in the above examples. Quantified cell lysates from leukemia samples and normal blood cells are then resolved on SDS-PAGE and prepared for Western blotting to probe for KIF20A and other biomarkers.

### 3. Results

KIF20A expression is significantly increased in cell and fluid samples obtained from leukemia patients as compared to expression in cell and fluid samples isolated from normal subjects. All normal subjects show nearly undetectable or nearly undetectable levels of KIF20A protein expression, while samples obtained from leukemia patients show detectable levels of KIF20A.

### Example 16

### Preparation and Use of Focused Microarray to Detect KIF20A in Samples Obtained from Normal Subjects and Leukemia Patients

1. Total RNA Isolation and cDNA Labeling

Patient marrow tissues and blood are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA group in order to compare them together.

Blood samples are prepared as described in Example 16. For leukemia tissue samples, human marrow tissues are homogenized and prepared for analysis following procedures described in Example 10.

First strand cDNA labeling, cDNA digestion, capture probe preparation and focused microarray preparation are accomplished using procedures described in Example 1. In addition, quality control and focused microarray hybridization are performed according to procedures described in Example 1. The QuantArray® data results are analyzed according to the procedures described above in Example 1.

### 2. Results

KIF20A mRNA expression correlates with KIF20A protein expression. Increased levels of KIF20A mRNA are detected in cell and fluid samples obtained patients suffering from leukemia compared to expression in samples from normal subjects. Cell and fluid samples from patients suffering from leukemia have higher levels of KIF20A mRNA expression than do samples from normal subjects.

### Example 17

### Western Blot Analysis of Samples Isolated from Sarcoma Patients and Normal Subjects

### 1. Patient Samples and Normal Samples

Patient tissue samples are obtained from Asterand, Inc. (Detroit, Mich.), Clinomics Biosciences, Inc (Watervliet, N.Y.) and Biochain Institute, Inc. (Hayward, Calif.). The samples are isolated from normal Sarcoma and Sarcoma cancer samples, and are frozen into blocks of tissue. Protein cell extracts are then prepared from each block. Each patient included in the study is screened against the same normal total RNA group in order to compare them together. The tumor group is composed of at least 20 cases. The Prostate normal group is composed of at least 20 cases.

### 2. Western Blot Analysis of KIF20A in Sarcoma Cancer and Sarcoma Normal Samples

Sample preparation and Western blot analysis are performed as described in Example 8.

### 3. Results

KIF20A expression is increased in tumor samples obtained from sarcoma tumor patients compared to expression in normal samples isolated from normal subjects. All normal subjects show nearly undetectable or undetectable levels of KIF20A protein expression, while samples obtained from sarcoma cancer patients show detectable levels of KIF20A.

### Example 18

### ELISA Analysis of KIF20A in Sarcoma Cancer and Normal Tissues

### 1. Isolation and Preparation of Patient and Normal Tissues

Patient tissue samples are obtained and are prepared as described in Example 6.

### 2. ELISA Analysis

ELISA analysis is performed as described in Example 9.

### 3. Results

ELISA results show that samples from normal subjects expressed less KIF20A protein compared to samples from sarcoma cancer patients. These results confirm the results obtained by the Western blot analysis.

### Example 19

### Focused Microarray to Detect KIF20A in Samples Obtained from Normal Sarcoma Subjects and Sarcoma Cancer Patients

1. Total RNA Isolation and cDNA Labeling

Patient Sarcoma tissue samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA group in order to compare them together.

### 2. Capture Probe and Focused Microarray Preparation

Capture probe preparation and printing of capture probes are performed according to the procedure provided in Example 12. The preparation of the microarray, quality control, hybridization, and analysis of the results are performed as described in Example 14.

### 3. Results

KIF20A mRNA expression correlates with KIF20A protein expression. Increased levels of KIF20A mRNA are detected in cell sample obtained patients suffering from sarcoma cancer compared to expression in samples from normal subjects. Cell samples from patients suffering from sarcoma cancer have higher levels of KIF20A mRNA expression than do normal subjects.

### Example 20

### Real-Time PCR Analysis of Samples Isolated from Sarcoma Cancer Patients and Normal Subjects

### 1. Patient Samples and RNA Isolation

Total RNA extraction from tumor cell lines and patient samples is performed as described in Example 5.

### 2. Real-Time PCR

Real-time PCR and analysis of results are performed as shown in Example 6.

### 3. Results

Increased levels of RNA expression are identified in colon tumor samples compared to normal colon samples. Normal sarcoma samples show less RNA expression of KIF20A than do sarcoma tumor samples. These results confirm the results obtained from the microarray experiments described in Example 23.

### Example 21

### Western Blot Analysis of Samples Isolated from Melanoma Patients and Normal Subjects

### 1. Patient Samples and Normal Samples

Patient tissues and fluid samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA group in order to compare them together.

### 2. Western Blot Analysis of KIF20A in Melanoma and Normal Samples

Sample preparation and Western blot analysis are performed as described in Example 12.

### 3. Results

KIF20A expression is increased in samples obtained from melanoma tumor patients compared to samples isolated from normal subjects. All normal subjects show undetectable or nearly undetectable levels of KIF20A protein expression, while samples obtained from melanoma cancer patients show detectable levels of KIF20A.

### Example 22

### ELISA Analysis of KIF20A in Melanoma Cancer and Melanoma Normal Tissues

### 1. Isolation and Preparation of Patient and Normal Tissues

Patient tissue samples are obtained and are prepared as described in Example 6.

### 2. ELISA Analysis

ELISA analysis is performed as described in Example 9

### 3. Results

ELISA results show that normal subjects expressed less KIF20A protein compared to melanoma cancer patient samples. These results confirm the results obtained in the Western blot analysis.

### Example 23

### Preparation and Use of Focused Microarray to Detect KIF20A in Samples Obtained from Normal Melanoma Subjects and Melanoma Cancer Patients

1. Total RNA Isolation and cDNA Labeling

Patient Melanoma tissue samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA group in order to compare them together.

### 2. Capture Probe and Focused Microarray Preparation

Capture probe preparation and printing of capture probes are performed according to the procedure provided in Example 15. The preparation of the microarray, quality control, hybridization, and analysis of the results is performed as detailed in Example 14.

### 3. Results

KIF20A mRNA expression correlates with KIF20A protein expression. Increased levels of KIF20A mRNA are detected in cell obtained patients suffering from melanoma cancer compared to normal subjects. Cell samples from patients suffering from melanoma cancer have higher levels of KIF20A mRNA expression than are found in samples from normal subjects.

### Example 24

### Real-Time PCR Analysis of Samples Isolated from Melanoma Cancer Patients and Normal Melanoma Subjects

### 1. Patient Samples and RNA Isolation

Total RNA extraction from tumor cell lines and patient samples is performed as described in Example 8.

### 2. Real-Time PCR

Real-time PCR and analysis of results is performed as described in Example 6.

### 3. Results

Increased levels of RNA expression are identified in colon tumor samples compared to expression in normal colon samples. Normal melanoma samples show less KIF20A RNA expression than do melanoma tumor samples. These results confirm the results obtained from the microarray experiments described in Example 27.

### EQUIVALENTS

Those skilled in the art will recognize, or be able to ascertain, using no more than routine experimentation, numerous equivalents to the specific compositions and procedures described herein. Such equivalents are considered to be within the scope of this invention, and are covered by the following claims.

1. A method for detecting a neoplasm comprising:
a) obtaining a potentially neoplastic test sample and a corresponding non-neoplastic control sample;
b) detecting a level of KIF20A expression in the test sample and in the control sample; and
c) comparing the level of KIF20A expression in the test sample to the level of KIF20A expression in the control sample,
the test sample being neoplastic if the level of KIF20A expression in the test sample is detectably greater than the level of KIF20A expression in the control sample.

2. The method of claim 1, wherein the neoplastic test sample and the control samples are cell samples of the same lineage.

3. The method of claim 2, wherein detecting the level of expression of KIF20A comprises isolating a cytoplasmic fraction from the test cell sample and from the control cell sample, and then separately detecting the level of expression of KIF20A in these cytoplasmic fractions.

4. The method of claim 1, wherein the level of expression of KIF20A protein is detected by contacting the test sample and the control sample with a KIF20A-specific protein binding agent selected from the group consisting of an anti-KIF20A antibody, KIF20A-binding portions of an antibody, KIF20A-specific ligands, KIF20A-specific aptamers, and KIF20A inhibitors.

5. The method of claim 4, wherein the KIF20A-specific protein binding agent is immobilized on a solid support.

6. The method of claim 1, wherein KIF20A expression is detected by detecting the level of expression of KIF20A RNA by contacting the test sample and the control sample with a KIF20A RNA-specific nucleic acid binding agent and determining how much of the nucleic acid binding agent is hybridized to KIF20A RNA in the test sample and in the control sample.

7. The method of claim 6, wherein the nucleic acid binding agent is immobilized on a solid support.

8. The method of claim 1, wherein the level of expression of KIF20A in the test sample is at least 1.5, at least 2, at least 4, at least 6, at least 8, at least 10, or at least 20 times greater than the level of expression of KIF20A in the control sample.

9. The method of claim 1, wherein the test sample is isolated from a patient suffering from ovarian cancer.

10. The method of claim 1, wherein the test sample is isolated from a patient suffering from breast cancer.

11. The method of claim 1, wherein the test sample is isolated from a patient suffering from colon cancer.

12. The method of claim 1, wherein the test sample is isolated from a patient suffering from lung cancer.

13. The method of claim 1, wherein the test sample is isolated from a patient suffering from melanoma.

14. The method of claim 1, wherein the test sample is isolated from a patient suffering from sarcoma.

15. The method of claim 1, wherein the test sample is isolated from a patient suffering from leukemia.

16. The method of claim 1, wherein the test sample and the control samples are fluid samples.

17. The method of claim 16, wherein the level of KIF20A protein expression is determined by measuring the level of anti-KIF20A antibody in the test fluid sample and in the control fluid sample.

18. The method of claim 17, wherein the test and control fluid samples are serum samples.

19. The method of claim 17, wherein the level of expression of anti-KIF20A antibody is detected with an anti-KIF20A antibody-specific antibody, or anti-KIF20A antibody-specific antibody fragment thereof.

20. A method for detecting a neoplasm comprising:
a) obtaining a potentially neoplastic test sample and a non-neoplastic control sample;
b) detecting a level of KIF20A expression in the test sample and in the control sample;
c) detecting a level of expression of at least one of TRIM59, TTK, SLC7A5, and/or UHRF1; and
d) comparing the level of KIF20A expression and the level of expression of at least one of TTK, SLC7A5, TRIM59 and/or UHRF1 in the test sample to the level of KIF20A expression and the level of expression of the at least one of TTK, SLC7A5, TRIM59 and/or UHRF1 in the control sample,
the test sample being neoplastic if the levels of expression of KIF20A and the at least one of TTK, SLC7A5, TRIM59 and/or UHRF1 in the test sample are detectably greater than the levels of expression of KIF20A and the at least one of TTK, SLC7A5, TRIM59 and/or UHRF1 in the control sample.

21. The method of claim 20, wherein detecting step (c) comprises detecting the level of expression of at least UHRF1, and comparing step (d) comprises comparing the levels of expression of KIF20A and at least UHRF1 in the test and control samples.

22. The method of claim 20, wherein detecting step (c) comprises detecting the level of expression of at least TTK, and comparing step (d) comprises comparing the levels of expression of KIF20A and at least TTK in the test and control samples.

23. The method of claim 21, wherein detecting step (c) further comprises detecting the level of expression of at least TTK, and comparing step (d) further comprises comparing the levels of expression of TTK in the test and control samples.

24. The method of claim 20, wherein the level of KIF20A expression is detected by contacting the test sample and the control sample with a KIF20A-specific protein binding agent selected from the group consisting of an KIF20A-specific antibody, KIF20A-specific binding portions of an antibody, a KIF20A-specific ligand, a KIF20A-specific aptamer, and an KIF20A inhibitor.

25. The method of claim 24, wherein the KIF20A-specific protein binding agent is immobilized on a solid support.

26. The method of claim 20, wherein the level of expression of KIF20A in the test and control samples is measured by measuring the level of KIF20A RNA and the level of at least one of TTK RNA, SLC7A5 RNA, TRIM59 RNA, and/or UHRF1 RNA in the test and control samples.

27. The method of claim 26, wherein the level of expression of KIF20A RNA and the level of expression of at least one of TTK RNA, SLC7A5 RNA, TRIM59 RNA, and/or UHRF1 RNA are detected by contacting the test sample and the control sample with an KIF20A-specific nucleic acid binding agent and with at least one of a TTK-specific nucleic acid binding agent, a SLC7A5-specific nucleic binding agent, a TRIM59-specific nucleic acid binding agent, and a UHRF1-specific nucleic acid binding agent.

28. The method of claim 20, wherein the levels of expression of KIF20A, TTK, SLC7A5, TRIM59 and/or KIF20A in the test sample are at least 1.5 times greater than the level of expression of KIF20A, TTK, SLC7A5, TRIM59, and/or UHRF1 in the control sample.

29. The method of claim 20, wherein the test and control samples are cell samples.

30. The method of claim 29, wherein detecting the level of expression of KIF20A and the level of expression of at least one of TTK, SLC7A5, TRIM59 and/or UHRF1 comprises isolating a cytoplasmic fraction from the test cell sample and from the control cell sample, and then detecting the levels of expression of KIF20A and at least one of TTK, SLC7A5, TRIM59 and/or UHRF1 in each of these cytoplasmic fractions.

31. The method of claim 20, wherein the test and control samples are fluid samples.

32. The method of claim 31, wherein the level of expression of KIF20A is measured by detecting a level of anti-KIF20A antibody in a test fluid sample and in a control fluid sample.

33. The method of claim 20, wherein the test sample is isolated from a tissue of a patient suffering from ovarian cancer, breast cancer, lung cancer, sarcoma, melanoma, or leukemia.

34. A kit for diagnosing or detecting neoplasia, comprising:
a) a first probe specific for the detection of KIF20A; and
b) a second probe specific for the detection of a neoplasia marker selected from the group consisting of TTK, SLC7A5, TRIM59, UHRF1, and combinations thereof,


SEQUENCE LISTING (10 sequences)

SEQ ID NO: 1 - protein, 890 aa, Homo sapiens
MSQGILSPPAGLLSDDDVVVSPMFESTAADLGSVVRKNLLSDCSVVSTSLEDKQQVPSEDSMEKVKVYLRVRPLLPSELERQEDQGCVRIENVETLVLQAPKDSFALKSNERGIGQATHRFTFSQIFGPEVGQASFFNLTVKEMVKDVLKGQNWLIYTYGVTNSGKTHTIQGTIKDGGILPRSLALIFNSLQGQLHPTPDLKPLLSNEVIWLDSKQIRQEEMKKLSLLNGGLQEEELSTSLKRSVYIESRIGTSTSFDSGIAGLSSISQCTSSSQLDETSHRWAQPDTAPLPVPANIRFSIWISFFEIYNELLYDLLEPPSQQRKRQTLRLCEDQNGNPYVKDLNWIHVQDAEEAWKLLKVGRKNQSFASTHLNQNSSRSHSIFSIRILHLQGEGDIVPKISELSLCDLAGSERCKDQKSGERLKEAGNINTSLHTLGRCIAALRQNQQNRSKQNLVPFRDSKLTRVFQGFFTGRGRSCMIVNVNPCASTYDETLHVAKFSAIASQLVHAPPMQLGFPSLHSFIKEHSLQVSPSLEKGAKADTGLDDDIENEADISMYGKEELLQVVEAMKTLLLKERQEKLQLEMHLRDEICNEMVEQMQQREQWCSEHLDTQKELLEEMYEEKLNILKESLTSFYQEEIQERDEKIEELEALLQEARQQSVAHQQSGSELALRRSQRLAASASTQQLQEVKAKLQQCKAELNSTTEELHKYQKMLEPPPSAKPFTIDVDKKLEEGQKNIRLLRTELQKLGESLQSAERACCHSTGAGKLRQALTTCDDILIKQDQTLAELQNNMVLVKLDLRKKAACIAEQYHTVLKLQGQVSAKKRLGTNQENQQPNQQPPGKKPFLRNLLPRTPTCQSSTDCSPYARILRSRRSPLLKSGPFGKKY

SEQ ID NO: 2 - protein, 403 aa, Homo sapiens
MHNFEEELTCPICYSIFEDPRVLPCSHTFCRNCLENILQASGNFYIWRPLRIPLKCPNCRSITEIAPTGIESLPVNFALRAIIEKYQQEDHPDIVTCPEHYRQPLNVYCLLDKKLVCGHCLTIGQHHGHPIDDLQSAYLKEKDTPQKLLEQLTDTHWTDLTHLIEKLKEQKSHSEKMIQGDKEAVLQYFKELNDTLEQKKKSFLTALCDVGNLINQEYTPQIERMKEIREQQLELMALTISLQEESPLKFLEKVDDVRQHVQILKQRPLPEVQPVEIYPRVSKILKEEWSRTEIGQIKNVLIPKMKISPKRMSCSWPGKDEKEVEFLKILNIVVVTLISVILMSILFFNQHIITFLSEITLIWFSEASLSVYQSLSNSLHKVKNILCHIFYLLKEFVWKIVSH

SEQ ID NO: 3 - protein, 857 aa, Homo sapiens
MESEDLSGRELTIDSIMNKVRDIKNKFKNEDLTDELSLNKISADTTDNSGTVNQIMMMANNPEDWLSLLLKLEKNSVPLSDALLNKLIGRYSQAIEALPPDKYGQNESFARIQVRFAELKAIQEPDDARDYFQMARANCKKFAFVHISFAQFELSQGNVKKSKQLLQKAVERGAVPLEMLEIALRNLNLQKKQLLSEEEKKNLSASTVLTAQESFSGSLGHLQNRNNSCDSRGQTTKARFLYGENMPPQDAEIGYRNSLRQTNKTKQSCPFGRVPVNLLNSPDCDVKTDDSVVPCFMKRQTSRSECRDLVVPGSKPSGNDSCELRNLKSVQNSHFKEPLVSDEKSSELIITDSITLKNKTESSLLAKLEETKEYQEPEVPESNQKQWQSKRKSECINQNPAASSNHWQIPELARKVNTEQKHTTFEQPVFSVSKQSPPISTSKWFDPKSICKTPSSNTLDDYMSCFRTPVVKNDFPPACQLSTPYGQPACFQQQQHQILATPLQNLQVLASSSANECISVKGRIYSILKQIGSGGSSKVFQVLNEKKQIYAIKYVNLEEADNQTLDSYRNEIAYLNKLQQHSDKIIRLYDYEITDQYIYMVMECGNIDLNSWLKKKKSIDPWERKSYWKNMLEAVHTIHQHGIVHSDLKPANFLIVDGMLKLIDFGIANQMQPDTTSVVKDSQVGTVNYMPPEAIKDMSSSRENGKSKSKISPKSDVWSLGCILYYMTYGKTPFQQIINQISKLHAIIDPNHEIEFPDIPEKDLQDVLKCCLKRDPKQRISIPELLAHPYVQIQTHPVNQMAKGTTEEMKYVLGQLVGLNSPNSILKAAKTLYEHYSGGESHNSSSSKTFEKKRGKK

SEQ ID NO: 4 - protein, 507 aa, Homo sapiens
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 5 - protein, 793 aa, Homo sapiens
MWIQVRTMDGRQTHTVDSLSRLTKVEELRRKIQELFHVEPGLQRLFYRGKQMEDGHTLFDYEVRLNDTIQLLVRQSLVLPHSTKERDSELSDTDSGCCLGQSESDKSSTHGEAAAETDSRPADEDMWDETELGLYKVNEYVDARDTNMGAWFEAQVVRVTRKAPSRDEPCSSTSRPALEEDVIYHVKYDDYPENGVVQMNSRDVRARARTIIKWQDLEVGQVVMLNYNPDNPKERGFWYDAEISRKRETRTARELYANVVLGDDSLNDCRIIFVDEVFKIERPGEGSPMVDNPMRRKSGPSCKHCKDDVNRLCRVCACHLCGGRQDPDKQLMCDECDMAFHIYCLDPPLSSVPSEDEWYCPECRNDASEVVLAGERLRESKKKAKMASATSSSQRDWGKGMACVGRTKECTIVPSNHYGPIPGIPVGTMWRFRVQVSESGVHRPHVAGIHGRSNDGAYSLVLAGGYEDDVDHGNFFTYTGSGGRDLSGNKRTAEQSCDQKLTNTNRALALNCFAPINDQEGAEAKDWRSGKPVRVVRNVKGGKNSKYAPAEGNRYDGIYKVVKYWPEKGKSGFLVWRYLLRRDDDEPGPWTKEGKDRIKKLGLTMQYPEGYLEALANREREKENSKREEEEQQEGGFASPRTGKGKWKRKSAGGGPSRAGSPRRTSKKTKVEPYSLTAQQSSLIREDKSNAKLWNEVLASLKDRPASGSPFQLFLSKVEETFQCICCQELVFRPITTVCQHNVCKDCLDRSFRAQVFSCPACRYDLGRSYAMQVNQPLQTVLNQLFPGYGNGR

SEQ ID NO: 6 - DNA, 2972 bp, Homo sapiens
tcttcggacctaggctgccctgccgtcatgtcgcaagggatcctttctccgccagcgggcttgctgtccgatgacgatgtcgtagtttctcccatgtttgagtccacagctgcagatttggggtctgtggtacgcaagaacctgctatcagactgctctgtcgtctctacctccctagaggacaagcagcaggttccatctgaggacagtatggagaaggtgaaagtatacttgagggttaggcccttgttaccttcagagttggaacgacaggaagatcagggttgtgtccgtattgagaatgtggagacccttgttctacaagcacccaaggactcgtttgccctgaagagcaatgaacggggaattggccaagccacacacaggttcaccttttcccagatctttgggccagaagtgggacaggcatccttcttcaacctaactgtgaaggagatggtaaaggatgtactcaaagggcagaactggctcatctatacatatggagtcactaactcagggaaaacccacacgattcaaggtaccatcaaggatggagggattctcccccggtccctggcgctgatcttcaatagcctccaaggccaacttcatccaacacctgatctgaagcccttgctctccaatgaggtaatctggctagacagcaagcagatccgacaggaggaaatgaagaagctgtccctgctaaatggaggcctccaagaggaggagctgtccacttccttgaagaggagtgtctacatcgaaagtcggataggtaccagcaccagcttcgacagtggcattgctgggctctcttctatcagtcagtgtaccagcagtagccagctggatgaaacaagtcatcgatgggcacagccagacactgccccactacctgtcccggcaaacattcgcttctccatctggatctcattctttgagatctacaacgaactgctttatgacctattagaaccgcctagccaacagcgcaagaggcagactttgcggctatgcgaggatcaaaatggcaatccctatgtgaaagatctcaactggattcatgtgcaagatgctgaggaggcctggaagctcctaaaagtgggtcgtaagaaccagagctttgccagcacccacctcaaccagaactccagccgcagtcacagcatcttctcaatcaggatcctacaccttcagggggaaggagatatagtccccaagatcagcgagctgtcactctgtgatctggctggctcagagcgctgcaaagatcagaagagtggtgaacggttgaaggaagcaggaaacattaacacctctctacacaccctgggccgctgtattgctgcccttcgtcaaaaccagcagaaccggtcaaagcagaacctggttcccttccgtgacagcaagttgactcgagtgttccaaggtttcttcacaggccgaggccgttcctgcatgattgtcaatgtgaatccctgtgcatctacctatgatgaaactcttcatgtggccaagttctcagccattgctagccagcttgtgcatgccccacctatgcaactgggattcccatccctgcactcgttcatcaaggaacatagtcttcaggtatcccccagcttagagaaaggggctaaggcagacacaggccttgatgatgatattgaaaatgaagctgacatctccatgtatggcaaagaggagctcctacaagttgtggaagccatgaagacactgcttttgaaggaacgacaggaaaagctacagctggagatgcatctccgagatgaaatttgcaatgagatggtagaacagatgcaacagcgggaacagtggtgcagtgaacatttggacacccaaaaggaactattggaggaaatgtatgaagaaaaactaaatatcctcaaggagtcactgacaagtttttaccaagaagagattcaggagcgggatgaaaagattgaagagctagaagctctcttgcaggaagccagacaacagtcagtggcccatcagcaatcagggtctgaattggccctacggcggtcacaaaggttggcagcttctgcctccacccagcagcttcaggaggttaaagctaaattacagcagtgcaaagcagagctaaactctaccactgaagagttgcataagtatcagaaaatgttagaaccaccaccctcagccaagcccttcaccattgatgtggacaagaagttagaagagggccagaagaatataaggctgttgcggacagagcttcagaaacttggtgagtctctccaatcagcagagagagcttgttgccacagcactggggcaggaaaacttcgtcaagccttgaccacttgtgatgacatcttaatcaaacaggaccagactctggctgaactgcagaacaacatggtgctagtgaaactggaccttcggaagaaggcagcatgtattgctgagcagtatcatactgtgttgaaactccaaggccaggtttctgccaaaaagcgccttggtaccaaccaggaaaatcagcaaccaaaccaacaaccaccagggaagaaaccattccttcgaaatttacttccccgaacaccaacctgccaaagctcaacagactgcagcccttatgcccggatcctacgctcacggcgttcccctttactcaaatctgggccttttggcaaaaagtactaaggctgtggggaaagagaagagcagtcatggccctgaggtgggtcagctactctcctgaagaaataggtctcttttatgctttaccatatatcaggaattatatccaggatgcaatactcagacactagcttttttctcacttttgtattataaccacctatgtaatctcatgttgttgtttttttttatttacttatatgatttctatgcacacaaaaacagttatattaaagatattattgttcacattttttattgaaaaaaaaaaaaaa

SEQ ID NO: 7 - DNA, 3886 bp, Homo sapiens
ggcacctgaagcccttcggggcagaggagggcggggactcggggcggctctcagcatccgcctggagctcgtggcgctgtgtttccgtgctgtggagttgcctggtccgcttcctccccgcgaataagaataaaagattctggaggagttggagaagagtgtattcagcccccaaaccacgagatcaacaaagaaatgcacaattttgaggaagagttaacttgtcccatatgttatagtatttttgaagatcctcgtgtactgccatgctctcatacattttgtagaaattgtttggaaaacattcttcaggcatctggtaacttttatatatggagacctttacgaattccactcaagtgccctaattgcagaagtattactgaaattgctccaactggcattgaatctttacctgttaattttgcactaagggctattattgaaaagtaccagcaagaagaccatccagatattgtcacctgccctgaacattacaggcaaccattaaatgtttactgtctattagataaaaaattagtttgtggtcattgccttaccataggtcaacatcatggtcatcctatagatgaccttcaaagtgcctatttgaaagaaaaggacactcctcaaaaactgcttgaacagttgactgacacacactggacagatcttacccatcttattgaaaagctgaaagaacaaaaatctcattctgagaaaatgatccaaggcgataaggaagctgttctccagtattttaaggagcttaatgatacattagaacagaaaaaaaaaagtttcctaacggctctctgtgatgttggcaatctaattaatcaagaatatactccacaaattgaaagaatgaaggaaatacgagagcagcagcttgaattaatggcactgacaatatctttacaagaagagtctccacttaaatttcttgaaaaagttgatgatgtacgccagcatgtacagatcttgaaacaaagaccacttcctgaggttcaacccgttgaaatttatcctcgagtaagcaaaatattgaaagaagaatggagcagaacagaaattggacaaattaagaacgttctcattcccaaaatgaaaatttctccaaaaaggatgtcatgttcctggcctggtaaggatgaaaaggaagttgaatttttaaaaattttaaacattgttgtagttacattaatttcagtaatactgatgtcgatactctttttcaaccaacacatcataacctttttaagtgaaatcactttaatatggttttctgaagcctctctatctgtttaccaaagtttatctaacagtctgcataaggtaaagaatatactgtgtcacattttctatttgttgaaggaatttgtgtggaaaatagtttcccattgaaaatgtcaacctgaattgtttaaatgggcttattctgtacattgctaaacaaaaaatggggtagcatggataaagagcaaactaagctttattagtgctgcaactaatataaacaaatgtttatatttgttgcttcttttggttagcaatgatatgtcaaagttatatctgaaatagtcaaatctttgggaaacagaatctagtaacaatttgaaaagtaatacactatcccatttttattggctttatgatggttgacataatgttgctgtgacatttaaacattcttgtaccaatattgtcttttaccattattatatactgcagttaattggctttacagttctttatatatatagcaaaatcctgaaagaacatatacctttattttgatgtggcttgaagcttttgaatgggtgaataaggatgatagaaaggtttcaaaatcaagcaacaaagtcttaaagtgataaggcatggcttaaagatcttttgatcaaacatacctgtgtttgagatagatttaagagccctaaatgcttatcaccattcactccaaataaaactattgcttttggataactgttagagtaaagtggctttttaaaagaaatttttgagactgggtctcaccttgttgcccaggctggagtgtagttgtctggtcatgactcactgcagtttcgaccacccaggctcaatcgatcctcccgcctctgcctctggagcagctgggacaaggcacacacccccatgcctggctaattaaaaaaattgttttttgtagaaacgaggttttgccatattgcccaggttggtctcaaacttctgggctcaagagatctgcccaccttggcctcccaaagtgctgggattacagacgttagccacactgtgcctgggggccagcattttctaatacttgtcatattctatagtttgtgcaaatttaagattgtttttttttctgctcgtcagtcaaatcagttcttggattaaaaactcattcttattagaacagaatcatgttggtaacttggtctgcaacaggttttgatggcatcatgtggactttattcatcttaactcatttaaattttctaccacattcccttaagctaatgcaaaagtaccaacaacttaatcttttttttttttttttttttttttttttgagacggagtcttgctctgtcgcccaggctggagtgcagtggcgtgatctcggctcattgcaagccccgcctcccgggttcacaccattctcctgcctcagcctcccgagtagctgggactacaggcatctgctaccacgcccagctaattttctgtatttttagtagagatggggtttcactgtgttagccaggatggtctctatctcctgacctcatgatccactcgcctcagcctcccaaaatgctgggattataggcgtgagccgccacgcccggcccaacaacttaatcttttattagctttgcttaagagggccaattaaatcaaagccctttagttccctttaacagggactggagttatgatgcatgtgttacgacttttggcccactgtccatgcaactctaagtgcaggttgatttgctttccagaaatcccaaaggggctgctcttggatcaccgaagagccttacctatatcaaatcaaaaagacattctgggtcagattagactatgctcctggccctacagattgcacataaactatcataaatacagctttttcagggaactagttctaaaactcttacctgctgagaataagtcttaacactaagatgactgtattatatcaatttattattagaatcagacttatacctagcacaattaactattgtgtgggcaaagaacatttaaagggcatagtagaggtaaggagagacacatactcagctagagataaaaatactaagttgcactgattacttaaaatactagtcacaccataaaagtgccctgtagtttcaaaaacacgtaagcaatgaaatgttagccattatgtgttaaactacttaatctcatttctgtgatgtgaatatttttaaccccctttttgtagatgagggaactgacaagtgacttgtccaaggccatatagctgtgagaaaacccatgcattctcttttcagagttcatgctatcactcaactttaaagtaggccaagattaatgttggtaagggtttgtaatctgtaagaatgctaaaaacgtaagtatatatatcattttagatttgacattttgtatcttgccagtttttaggagaacttttcattttgttaagtatgcatgaatatagttgagtatatgagtaactggttcttatgctgctgttttgtatttttaccagcaggaagattgcaaaagttgatgtatgtaaatcttgaaatatttctaagttttatgtataacaaaatatgtattttaataaacttcttttgatattttaaaaaaaaaaa

SEQ ID NO: 8 - DNA, 2984 bp, Homo sapiens
ggaaattcaaacgtgtttgcggaaaggagtttgggttccatcttttcatttccccagcgcagctttctgtagaaatggaatccgaggatttaagtggcagagaattgacaattgattccataatgaacaaagtgagagacattaaaaataagtttaaaaatgaagaccttactgatgaactaagcttgaataaaatttctgctgatactacagataactcgggaactgttaaccaaattatgatgatggcaaacaacccagaggactggttgagtttgttgctcaaactagagaaaaacagtgttccgctaagtgatgctcttttaaataaattgattggtcgttacagtcaagcaattgaagcgcttcccccagataaatatggccaaaatgagagttttgctagaattcaagtgagatttgctgaattaaaagctattcaagagccagatgatgcacgtgactactttcaaatggccagagcaaactgcaagaaatttgcttttgttcatatatcttttgcacaatttgaactgtcacaaggtaatgtcaaaaaaagtaaacaacttcttcaaaaagctgtagaacgtggagcagtaccactagaaatgctggaaattgccctgcggaatttaaacctccaaaaaaagcagctgctttcagaggaggaaaagaagaatttatcagcatctacggtattaactgcccaagaatcattttccggttcacttgggcatttacagaataggaacaacagttgtgattccagaggacagactactaaagccaggtttttatatggagagaacatgccaccacaagatgcagaaataggttaccggaattcattgagacaaactaacaaaactaaacagtcatgcccatttggaagagtcccagttaaccttctaaatagcccagattgtgatgtgaagacagatgattcagttgtaccttgttttatgaaaagacaaacctctagatcagaatgccgagatttggttgtgcctggatctaaaccaagtggaaatgattcctgtgaattaagaaatttaaagtctgttcaaaatagtcatttcaaggaacctctggtgtcagatgaaaagagttctgaacttattattactgattcaataaccctgaagaataaaacggaatcaagtcttctagctaaattagaagaaactaaagagtatcaagaaccagaggttccagagagtaaccagaaacagtggcaatctaagagaaagtcagagtgtattaaccagaatcctgctgcatcttcaaatcactggcagattccggagttagcccgaaaagttaatacagagcagaaacataccacttttgagcaacctgtcttttcagtttcaaaacagtcaccaccaatatcaacatctaaatggtttgacccaaaatctatttgtaagacaccaagcagcaataccttggatgattacatgagctgttttagaactccagttgtaaagaatgactttccacctgcttgtcagttgtcaacaccttatggccaacctgcctgtttccagcagcaacagcatcaaatacttgccactccacttcaaaatttacaggttttagcatcttcttcagcaaatgaatgcatttcggttaaaggaagaatttattccattttaaagcagataggaagtggaggttcaagcaaggtatttcaggtgttaaatgaaaagaaacagatatatgctataaaatatgtgaacttagaagaagcagataaccaaactcttgatagttaccggaacgaaatagcttatttgaataaactacaacaacacagtgataagatcatccgactttatgattatgaaatcacggaccagtacatctacatggtaatggagtgtggaaatattgatcttaatagttggcttaaaaagaaaaaatccattgatccatgggaacgcaagagttactggaaaaatatgttagaggcagttcacacaatccatcaacatggcattgttcacagtgatcttaaaccagctaactttctgatagttgatggaatgctaaagctaattgattttgggattgcaaaccaaatgcaaccagatacaacaagtgttgttaaagattctcaggttggcacagttaattatatgccaccagaagcaatcaaagatatgtcttcctccagagagaatgggaaatctaagtcaaagataagccccaaaagtgatgtttggtccttaggatgtattttgtactatatgacttacgggaaaacaccatttcagcagataattaatcagatttctaaattacatgccataattgatcctaatcatgaaattgaatttcccgatattccagagaaagatcttcaagatgtgttaaagtgttgtttaaaaagggacccaaaacagaggatatccattcctgagctcctggctcatccctatgttcaaattcaaactcatccagttaaccaaatggccaagggaaccactgaagaaatgaaatatgttctgggccaacttgttggtctgaattctcctaactccattttgaaagctgctaaaactttatatgaacactatagtggtggtgaaagtcataattcttcatcctccaagacttttgaaaaaaaaaggggaaaaaaatgatttgcagttattcgtaatgtcaaataccacctataaaatatattggactgttatactcttgaatccctgtggaaatctacatttgaagacaacatcactctgaagtgttatcagcaaaaaaaattcagtagattatctttaaaagaaaactgtaaaaatagcaaccacttatggtactgtatatattgtagacttgttttctctgttttatgctcttgtgtaatctacttgacatcattttactcttggaatagtgggtggatagcaagtatattctaaaaaactttgtaaataaagttttgtggctaaaatgacactaaaaaaaaaaaaaaaaaa

SEQ ID NO: 9 - DNA, 4543 bp, Homo sapiens
cggcgggcggcgcgcacactgctcgctgggccgcggctcccgggtgtcccaggcccggccggtgcgcagagcatggcgggtgcgggcccgaagcggcgcgcgctagcggcgccggcggccgaggagaaggaagaggcgcgggagaagatgctggccgccaagagcgcggacggctcggcgccggcaggcgagggcgagggcgtgaccctgcagcggaacatcacgctgctcaacggcgtggccatcatcgtggggaccattatcggctcgggcatcttcgtgacgcccacgggcgtgctcaaggaggcaggctcgccggggctggcgctggtggtgtgggccgcgtgcggcgtcttctccatcgtgggcgcgctctgctacgcggagctcggcaccaccatctccaaatcgggcggcgactacgcctacatgctggaggtctacggctcgctgcccgccttcctcaagctctggatcgagctgctcatcatccggccttcatcgcagtacatcgtggccctggtcttcgccacctacctgctcaagccgctcttccccacctgcccggtgcccgaggaggcagccaagctcgtggcctgcctctgcgtgctgctgctcacggccgtgaactgctacagcgtgaaggccgccacccgggtccaggatgcctttgccgccgccaagctcctggccctggccctgatcatcctgctgggcttcgtccagatcgggaagggtgatgtgtccaatctagatcccaacttctcatttgaaggcaccaaactggatgtggggaacattgtgctggcattatacagcggcctctttgcctatggaggatggaattacttgaatttcgtcacagaggaaatgatcaacccctacagaaacctgcccctggccatcatcatctccctgcccatcgtgacgctggtgtacgtgctgaccaacctggcctacttcaccaccctgtccaccgagcagatgctgtcgtccgaggccgtggccgtggacttcgggaactatcacctgggcgtcatgtcctggatcatccccgtcttcgtgggcctgtcctgcttcggctccgtcaatgggtccctgttcacatcctccaggctcttcttcgtggggtcccgggaaggccacctgccctccatcctctccatgatccacccacagctcctcacccccgtgccgtccctcgtgttcacgtgtgtgatgacgctgctctacgccttctccaaggacatcttctccgtcatcaacttcttcagcttcttcaactggctctgcgtggccctggccatcatcggcatgatctggctgcgccacagaaagcctgagcttgagcggcccatcaaggtgaacctggccctgcctgtgttcttcatcctggcctgcctcttcctgatcgccgtctccttctggaagacacccgtggagtgtggcatcggcttcaccatcatcctcagcgggctgcccgtctacttcttcggggtctggtggaaaaacaagcccaagtggctcctccagggcatcttctccacgaccgtcctgtgtcagaagctcatgcaggtggtcccccaggagacatagccaggaggccgagtggctgccggaggagcatgcgcagaggccagttaaagtagatcacctcctcgaacccactccggttccccgcaacccacagctcagctgcccatcccagtccctcgccgtccctcccaggtcgggcagtggaggctgctgtgaaaactctggtacgaatctcatccctcaactgagggccagggacccaggtgtgcctgtgctcctgcccaggagcagcttttggtctccttgggccctttttcccttccctcctttgtttacttatatatatattttttttaaacttaaattttgggtcaacttgacaccactaagatgattttttaaggagctgggggaaggcaggagccttcctttctcctgccccaagggcccagaccctgggcaaacagagctactgagacttggaacctcattgctaccacagacttgcactgaagccggacagctgcccagacacatgggcttgtgacattcgtgaaaaccaaccctgtgggcttatgtctctgccttagggtttgcagagtggaaactcagccgtagggtggcactgggagggggtgggggatctgggcaaggtgggtgattcctcccaggaggtgcttgaggccccgatggactcctgaccataatcctagccccgagacaccatcctgagccagggaacagccccagggttggggggtgccggcatctcccctagctcaccaggcctggcctctgggcagtgtggcctcttggctatttctgtgtccagttttggaggctgagttctggttcatgcagacaaagccctgtccttcagtcttctagaaacagagacaagaaaggcagacacaccgcggccaggcacccatgtgggcgcccaccctgggctccacacagcagtgtcccctgccccagaggtcgcagctaccctcagcctccaatgcattggcctctgtaccgcccggcagccccttctggccggtgctgggttcccactcccggcctaggcacctccccgctctccctgtcacgctcatgtcctgtcctggtcctgatgcccgttgtctaggagacagagccaagcactgctcacgtctctgccgcctgcgtttggaggcccctgggctctcacccagtccccacccgcctgcagagagggaactagggcaccccttgtttctgttgttcccgtgaatttttttcgctatgggaggcagccgaggcctggccaatgcggcccactttcctgagctgtcgctgcctccatggcagcagccagggacccccagaacaagaagaccccgcaggatccctcctgagctcggggggctctgccttctcaggccccgggcttcccttctccccagccagaggtggagccaagtggtccagcgtcactccagtgctcagctgtggctggaggagctggcctgtggcacagccctgagtgtcccaagccgggagccaacgaagccggacacggcttcactgaccagcggctgctcaagccgcaagctctcagcaagtgcccagtggagcctgccgcccccgcctgggcaccgggaccccctcaccatccagtgggcccggagaaacctgatgaacagtttggggactcaggaccagatgtccgtctctcttgcttgaggaatgaagacctttattcacccctgccccgttgcttcccgctgcacatggacagacttcacagcgtctgctcataggacctgcatccttcctggggacgaattccactcgtccaagggacagcccacggtctggaggccgaggaccaccagcaggcaggtggactgactgtgttgggcaagacctcttccctctgggcctgttctcttggctgcaaataaggacagcagctggtgccccacctgcctggtgcattgctgtgtgaatccaggaggcagtggacatcgtaggcagccacggccccgggtccaggagaagtgctccctggaggcacgcaccactgcttcccactggggccggcggggcccacgcacgacgtcagcctcttaccttcccgcctcggctaggggtcctcgggatgccgttctgttccaacctcctgctctgggacgtggacatgcctcaaggatacagggagccggcggcctctcgacggcacgcacttgcctgttggctgctgcggctgtgggcgagcatgggggctgccagcgtctgttgtggaaagtagctgctagtgaaatggctggggccgctggggtccgtcttcacactgcgcaggtctcttctgggcgtctgagctggggtgggagctcctccgcagaaggttggtggggggtccagtctgtgatccttggtgctgtgtgccccactccagcctggggaccccacttcagaaggtaggggccgtgtcccgcggtgctgactgaggcctgcttccccctccccctcctgctgtgctggaattccacagggaccagggccaccgcaggggactgtctcagaagacttgatttttccgtccctttttctccacactccactgacaaacgtccccagcggtttccacttgtgggcttcaggtgttttcaagcacaacccaccacaacaagcaagtgcattttcagtcgttgtgcttttttgttttgtgctaacgtcttactaatttaaagatgctgtcggcaccatgtttatttatttccagtggtcatgctcagccttgctgctctgcgtggcgcaggtgccatgcctgctccctgtctgtgtcccagccacgcagggccatccactgtgacgtcggccgaccaggctggacaccctctgccgagtaatgacgtgtgtggctgggaccttctttattctgtgttaatggctaacctgttacactgggctgggttgggtagggtgttctggcttttttgtggggtttttatttttaaagaaacactcaatcatccta

SEQ ID NO: 10 - DNA, 4086 bp, Homo sapiens
ggccacttggcccgggcctcctttctcctctggtcgtggggaaggagggatgggttggaccttctgcttttctttcaattccctcttttcattctccttcctcctcaatcttcaacacttggctagtcgttaatgccttaagtgcttaatttgttgtgtctggtcctggccagggtctggctgtacaggaggactggaagggcatcctgggagtttcctggtgtccacaggccggacaaaagcaaccccgactccttagagcatggcatggctcagaggtgctggtaaaactgatgggggtttttgctgtccctcccctcagcgccgacaccatgtggatccaggttcggaccatggacgggaggcagacccacacggtggactcgctgtccaggctgaccaaggtggaggagctgaggcggaagatccaggagctgttccacgtggagccaggcctgcagaggctgttctacaggggcaaacagatggaggacggccataccctcttcgactacgaggtccgcctgaatgacaccatccagctcctggtccgccagagcctcgtgctcccccacagcaccaaggagcgggactccgagctctccgacaccgactccggctgctgcctgggccagagtgagtcagacaagtcctccacccacggtgaggcggccgccgagactgacagcaggccagccgatgaggacatgtgggatgagacggaattggggctgtacaaggtcaatgagtacgtcgatgctcgggacacgaacatgggggcgtggtttgaggcgcaggtggtcagggtgacgcggaaggccccctcccgggacgagccctgcagctccacgtccaggccggcgctggaggaggacgtcatttaccacgtgaaatacgacgactacccggagaacggcgtggtccagatgaactccagggacgtccgagcgcgcgcccgcaccatcatcaagtggcaggacctggaggtgggccaggtggtcatgctcaactacaaccccgacaaccccaaggagcggggcttctggtacgacgcggagatctccaggaagcgcgagaccaggacggcgcgggaactctacgccaacgtggtgctgggggatgattctctgaacgactgtcggatcatcttcgtggacgaagtcttcaagattgagcggccgggtgaagggagccccatggttgacaaccccatgagacggaagagcgggccgtcctgcaagcactgcaaggacgacgtgaacagactctgccgggtctgcgcctgccacctgtgcgggggccggcaggaccccgacaagcagctcatgtgcgatgagtgcgacatggccttccacatctactgcctggacccgcccctcagcagtgttcccagcgaggacgagtggtactgccctgagtgccggaatgatgccagcgaggtggtactggcgggagagcggctgagagagagcaagaagaaggcgaagatggcctcggccacatcgtcctcacagcgggactggggcaagggcatggcctgtgtgggccgcaccaaggaatgtaccatcgtcccgtccaaccactacggacccatcccggggatccccgtgggcaccatgtggcggttccgagtccaggtcagcgagtcgggtgtccatcggccccacgtggctggcatacacggccggagcaacgacggagcgtactccctagtcctggcggggggctatgaggatgatgtggaccatgggaattttttcacatacacgggtagtggtggtcgagatctttccggcaacaagaggaccgcggaacagtcttgtgatcagaaactcaccaacaccaacagggcgctggctctcaactgctttgctcccatcaatgaccaagaaggggccgaggccaaggactggcggtcggggaagccggtcagggtggtgcgcaatgtcaagggtggcaagaatagcaagtacgcccccgctgagggcaaccgctacgatggcatctacaaggttgtgaaatactggcccgagaaggggaagtccgggtttctcgtgtggcgctaccttctgcggagggacgatgatgagcctggcccttggacgaaggaggggaaggaccggatcaagaagctggggctgaccatgcagtatccagaaggctacctggaagccctggccaaccgagagcgagagaaggagaacagcaagagggaggaggaggagcagcaggaggggggcttcgcgtcccccaggacgggcaagggcaagtggaagcggaagtcggcaggaggtggcccgagcagggccgggtccccgcgccggacatccaagaaaaccaaggtggagccctacagtctcacggcccagcagagcagcctcatcagagaggacaagagcaacgccaagctgtggaatgaggtcctggcgtcactcaaggaccggccggcgagcggcagcccgttccagttgttcctgagtaaagtggaggagacgttccagtgtatctgctgtcaggagctggtgttccggcccatcacgaccgtgtgccagcacaacgtgtgcaaggactgcctggacagatcctttcgggcacaggtgttcagctgccctgcctgccgctacgacctgggccgcagctatgccatgcaggtgaaccagcctctgcagaccgtcctcaaccagctcttccccggctacggcaatggccggtgatctccaagcacttctcgacaggcgttttgctgaaaacgtgtcggagggctcgttcatcggcactgattttgttcttagtgggcttaacttaaacaggtagtgtttcctccgttccctaaaaaggtttgtcttcctttttttttttatttttatttttcaaatctatacattttcaggaatttatgtattctggctaaaagttggacttctcagtattgtgtttagttctttgaaaacataaaagcctgcaatttctcgacaaaacaacacaagattttttaaagatggaatcagaaactacgtggtgtggaggctgttgatgtttctggtgtcaagttctcagaagttgctgccaccaactctttaagaaggcgacaggatcagtccttctctcgggttctggcccccaaggtcagagcaagcatcttcctgacagcattttgtcatctaaagtccagtgacatggttccccgtggtggcccgtggcagcccgtggcatggcgtggctcagctgtctgttgaagttgttgcaaggaaaagaggaaacatctcgggcctagttcaaacctttgcctcaaagccatcccccaccagactgcttagcgtctgagatccgcgtgaaaagtcctctgcccacgagagcagggagttggggccacgcagaaatggcctcaaggggactctgctccacgtggggccaggcgtgtgactgacgctgtccgacgaaggcggccacggacggacgccagcacacgaagtcacgtgcaagtgcctttgattcgttccttctttctaaagacgacagtctttgttgttagcactgaattattgaaaatgtcaaccagattctagaaactgcggtcatccagttcttcctgacaccggatgggtgcttgggaaccgtttgagccttatagatcatttacattcaatttttttaactcagcaagtgagaacttacaagagggtttttttaaaatttttttttctcttaatgaacacattttctaaatgaattttttttgtagttactgtatatgtaccaagaaagatataacgttagggtttggttgtttttgtttttgtattttttttcttttgaaagggtttgttaatttttctaattttaccaaagtttgcagcctatacctcaataaaacagggatattttaaatcacatacctgcagacaaactggagcaatgttatttttaaagggtttttttcacctccttattcttagattattaatgtattagggaagaatgagacaattttgtgtaggctttttctaaagtccagtactttgtccagattttagattctcagaataaatgtttttcacagatagaaaaaaaaaaaaaaaaaaaaaa